(19) United States
(12) Patent Application Publication
(10) Pub. No.: US 2023/0295260 A1
(43) Pub. Date: Sep. 21, 2023

(54) GLP RECEPTOR AGONISTS

(71) Applicant: HEPTARES THERAPEUTICS LIMITED, Cambridge, Cambridgeshire (GB)

(72) Inventors: Giles Albert BROWN, Cambridge (GB); Miles Stuart CONGREVE, Cambridge (GB); Conor SCULLY, Cambridge (GB); Rebecca PAUL, Cambridge (GB); Andrea BORTOLATO, Cambridge (GB)

(73) Assignee: HEPTARES THERAPEUTICS LIMITED, Cambridge, Cambridgeshire (GB)

(21) Appl. No.: 17/909,907

(22) PCT Filed: Mar. 16, 2021
(86) PCT No.: PCT/GB2021/050657

§ 371 Date: Sep. 7, 2022

(30) Foreign Application Priority Data
Mar. 16, 2020 (GB) 2003762.8

(51) Int. Cl. C07K 14/605

(52) U.S. Cl. CPC C07K 14/605; A61K 38/00

(57) ABSTRACT

The disclosures herein relate to novel compounds of formula (1): and salts thereof, wherein Q, W, X, Y, Z, AA¹, AA², AA³, AA⁴, AA⁵, AA⁶, AA⁷, AA⁸, AA⁹, LysR, R ¹, R² and n are defined herein, and their use in treating, preventing, ameliorating, controlling or reducing the risk of disorders associated with Glucagon-like peptide (GLP) receptors.

This invention relates to a class of novel peptide compounds, their salts, pharmaceutical compositions containing them and their use in therapy of the human body. In particular, the invention is directed to a class of compounds which are agonists of Glucagon-like peptide (GLP) receptors. More particularly, the invention is directed to compounds that are agonists of the Glucagon-like peptide-1 (GLP-1) and Glucagon-like peptide-2 (GLP-2) receptors. More particularly, the invention is directed to compounds that are selective agonists of the Glucagon-like peptide-2 (GLP-2) receptor. The invention also relates to the manufacture and use of these compounds and compositions in the prevention or treatment of such diseases in which GLP receptors are involved.

### BACKGROUND OF THE INVENTION

Glucagon-like peptide-1 (GLP-1) and Glucagon-like peptide-2 (GLP-2) are highly conserved amino acid peptides that originate from the same precursor protein. These biologically active peptides are encoded by the proglucagon gene which undergoes tissue specific post-translational processing in the pancreas (alpha cells), intestine (L-cells) and the central nervous system (CNS). In the gastrointestinal tract, prohormone convertase ⅓ is responsible for cleaving proglucagon to give rise to a number of biologically active peptides including GLP-1, GLP-2, IP2, oxyntomodulin and glicentin. Both GLP-1 and GLP-2 are secreted in response to nutrient ingestion by intestinal L cells localised in the distal ileum and colon and plasma levels of these gut peptides are reported to be increased after food intake in man.

The actions of GLP-1 and GLP-2 are mediated through the activation of class B G protein coupled receptors, GLP-1 R and GLP-2R, which couple to the Gs protein and stimulate cAMP production via activation of adenylate cyclase. GLP-1 R is found expressed in the brain, pancreatic islet cells, heart, kidney and myenteric plexus neurones in the gastrointestinal tract. The expression of GLP-2R on the other hand, is more restricted, and the receptor is largely localised to the CNS and the gastrointestinal tract. A number of cell types have been reported to express GLP-2R in the gut including enteric neurons, subepithelial myofibroblasts and enteroendocrine cells, however the exact cellular distribution remains to be defined.

GLP-2 has been reported to be involved in a wide range of physiological functions including gut barrier function, mesenteric blood flow, gastric motility and acid secretion. Exogenous administration of GLP-2 stimulates crypt cell proliferation, enhances intestinal villi length and promotes the growth and repair of the small intestinal mucosa. The potent intestinotrophic activity of GLP-2 has been documented across species including rats, pigs and human. GLP-2 furthermore enhances intestinal absorptive capacity through regulation of intestinal brush border enzymes and solute carriers, highlighting the potential role of this gut hormone in the control of energy homeostasis. Based on the ability to promote potent intestinotrophic effects in the gut, Teduglutide, a GLP-2 analogue has been approved as pharmacological therapy for PN dependent SBS patients and has been shown to reduce PN requirements as well as promote enteral autonomy.

GLP-1 is a 31 amino acid peptide which is co-released with GLP-2 in response to luminal nutrients (carbohydrates, fats, proteins) and serves as a gut incretin hormone that works in concert with glucose-dependent insulinotropic polypeptide (GIP). GLP-1 plays a key physiological role in pancreatic islet β-cell function, regulating (3-cell proliferation as well as postprandial insulin synthesis/release. Studies have furthermore shown that GLP-1 controls the release of other gut peptides such as somatostatin and glucagon. Following its release, somatostatin acts to suppress GLP-1 and GIP secretion thereby establishing a feedback system in enteroendocrine cells. GLP-1 is a key anorexigenic peptide involved in the regulation of satiety and appetite control, and impacts Gl function through effects on gastric emptying and gut motility. Several GLP-1 agents are currently marketed for the treatment of type 2 diabetes mellitus and have been successful in improving glycemic control in diabetic patients.

Intestinal failure (IF) refers to a serious and disabling condition whereby the gut is unable to absorb necessary water, electrolytes, macro- and micronutrients for survival. The causes of IF are varied and can result from obstruction, dysmotility, surgical resection, congenital defect or disease associated loss of absorption.

Short bowel syndrome represents the most common cause of intestinal failure and arises from the physical or functional loss of a bowel section, often leading to malnutrition, weight loss, dehydration, diarrhoea, steatorrhoea, fatigue and abdominal pain. Management of SBS requires multidisciplinary care and parenteral nutrition (PN) support to compensate for the extensive fluid loss and to restore nutrient and electrolyte balances. Although critical for survival, long term dependence on parenteral nutrition can negatively impact the patient’s quality of life and furthermore increase the risk of life threatening complications such as catheter related sepsis, venous thrombosis and liver damage (e.g. steatosis, cholestasis).

The symptoms and severity of SBS can vary depending on the location and length of the remnant bowel. Intestinal motility is known to be influenced by multiple gut hormones including GLP-1, GLP-2 and PYY which are typically produced by L cells in the ileum and proximal colon. Hormones such as GLP-1 act to provide important feedback mechanisms to control the rate of Gl transit for efficient nutrient digestion and absorption. Patients with jejunostomy that have lost the ileal brake have lower fasting GLP-1 and GLP-2 concentrations in plasma and generally suffer rapid gastric emptying and Gl transit with high stoma output. Small pilot studies have demonstrated that exenatide or liraglutide (GLP-1 agonists) improve symptoms of diarrhoea in SBS patients and furthermore reduce the requirement for PN.

Adding to the complex clinical picture, evidence also exists for a dysregulated enteroinsular axis in patients with bowel resection that results in impaired insulin response in response to oral glucose administration. In addition, hyperglycemia is a frequent complication of parenteral nutrition in hospitalised patients and can increase the risk of death and infectious complications. The prevalence of hyperglycemia in patients receiving specialised nutritional support is estimated to be up to 30% for those receiving enteral nutrition and 50% in patients on parenteral nutrition. It is recognised that continued poor control of hyperglycemia can lead to a decline in pancreatic beta cell function and can contribute to exacerbating complications such as microvascular disease, cardiovascular events and hypertension. Patients with hyperglycemia during TPN are at greater risk of being admitted to ICU, have longer hospital stays and higher mortality rates compared to those without hyperglycemia.

Based on the known insulinotropic activity of GLP-1 agonists, activation of this mechanism could therefore potentially offer additional benefit in those that develop diminished insulin sensitivity post-surgery and in patients receiving parenteral nutrition. These findings therefore highlight the potential for a combined GLP-2/GLP-1 pharmacological approach in the management of intestinal failure conditions including SBS.

Other intestinal failure conditions where GLP-2/GLP-1 agonists could provide benefit include rare congenital diarrhoeal diseases such as Tufting enteropathy which presents with early onset severe intractable diarrhoea that persists during fasting. Acute treatment of infants with parenteral nutrition, fluid and electrolyte replacement is critically required to prevent dehydration, electrolyte imbalance and impaired growth resulting from severe malnutrition.

Gene encoding the epithelial cell adhesion molecule EpCAM shows association with Tufting enteropathy and to date over 25 EpCAM mutations have been described in the literature. Mutations in the EpCAM gene leads to the loss of cell surface expression, giving rise to the distinctive histological features in the intestinal epithelium, such as focal crowding of enterocytes and formation of ‘tufts’. Mice carrying deletion of exon 4 of the EpCAM gene demonstrate similar morphological defects to Tufting patients with significant morbidity and mortality. EpCAM directly associates with claudin 7, a tight junction molecule and disruptions of this gene leads to poor enterocyte adhesion and impaired gut barrier function, possibly through downregulation of tight junction molecules.

Infants with Tufting enteropathy have low IGF-1 levels and depend on parenteral nutrition to compensate for the diminished capacity to absorb nutrients. Currently there are no pharmacological treatments for this debilitating condition and there is pressing need for agents that can improve intestinal function to promote independence from parenteral feeding. Recent analysis of the long term outcome of Tufting patients has revealed that enteral autonomy can successfully be achieved in most patients if they are effectively managed under specialist care settings. Therapies that promote early weaning are expected to lead to a better long term outcome in these patients and improve the quality of life. Agents acting at GLP-2 and GLP-1 receptors may hold promise in repairing barrier function and aiding recovery of intestinal function in this congenital diarrhoeal disease.

### SUMMARY OF THE INVENTION

The present invention relates to novel compounds with agonist activity at the GLP-2 and GLP-1 receptor, pharmaceutical compositions comprising these, and use of the compounds for the manufacture of medicaments for treatment of diseases.

Accordingly, in one embodiment the invention provides a compound of the formula (1):

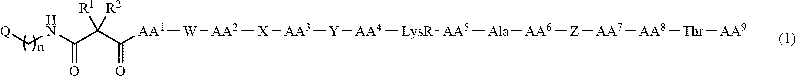

wherein;
Q is phenyl or a monocyclic heteroaryl ring each of which may be optionally substituted with one or more R^{q} groups;
R^{q} is selected from halogen, hydroxyl, amino or C₁₋₆ alkyl having an alkyl chain optionally containing one or more heteroatoms selected from O, N, or S;
n is 1 to 3;
R¹ and R² are independently selected from hydrogen or a C₁₋₆ alkyl group, or together with the carbon to which they are attached join to form a C₃₋₈ cycloalkyl or a heterocyclyl group; W is a sequence —Gly—Ser—, —Ala—Ser— or —DAla—Ser—;
X is a sequence —Ser—Asp—Glu—Nle—DPhe—Thr— or —Ser—Asp—Glu—Nle—Asn—Thr—; Y is a sequence —Leu—Asp—;
Z is a sequence —Asp— Phe—lle—Asn—Trp—Leu—lle—GIn—Thr—;
AA¹ is -NHCHR³CO-; wherein R³ is selected from -(CH₂)_{y}CONH₂, -(CH₂)_{y}COOH or -(CH₂)_{y}tetrazolyl; where y is 1 or 2;
AA² is -NHCR^{4a}R^{4b}CO-; wherein R^{4a} is hydrogen or a C₁₋₃ alkyl group; and R^{4b} is a benzyl group optionally substituted with one or more halogen groups, C₁₋₃ alkyl groups or C₁₋₃ alkoxy groups;
AA³ is -Aib- or -lle-;
AA⁴ is -NHCR^{5a}R^{5b}CO-; wherein R^{5a} is hydrogen or a C₁₋₃ alkyl group; and R^{5b} is an optionally substituted C₁₋₆ alkyl group, or -(CH₂)ₓCONH₂; where x is 1 or 2;
AA⁵ is —Ala— or -Aib-;
AA⁶ is —Lys—, -Aib- or a group —LysR—;
AA⁷ is —Lys— or —Arg—;
AA⁸ is -NHCR^{6a}R^{6b}CO-, wherein R^{6a} is hydrogen or a C₁₋₃ alkyl group; and R^{6b} is an optionally substituted C₁₋₆ alkyl group;
AA⁹ is -NHCR^{7a}R^{7b}CO-; wherein R^{7a} is hydrogen or a C₁₋₃ alkyl group; and R^{7b} is -(CH₂)_{Z}COOH, or a benzyl group optionally substituted with one or more halogen groups, C₁₋₃ alkyl groups or C₁₋₃ alkoxy groups; where z is 1 or 2;
LysR is an N-substituted Lysine residue;
wherein the AA⁹ C-terminus is a carboxyl group or a carboxamide group, or is adjoined to any natural or non-natural amino acid sequence or any other moiety, functional group or groups;
or a tautomeric or stereochemically isomeric form thereof or a prodrug, salt or zwitterion thereof.

The GLP-2/GLP-1 derivatives of this invention can be used in the treatment of various diseases as described below.

In one aspect, the present invention provides a method for promoting growth of small bowel tissue in a patient in need thereof, comprising the step of delivering to the patient an intestinotrophic amount of a GLP-2/GLP-1 analogue of the present invention.

In a further aspect the present invention relates to a method for the preparation of a medicament for the treatment of gastrointestinal diseases that include intestinal failure or other conditions leading to nutrient malabsorption and intestinal insufficiency. Examples of such diseases may include small bowel syndrome, diarrhoeal diseases, inflammatory bowel syndrome, Crohn’s disease, Ulcerative colitis, pouchitis, radiation induced bowel damage, Celiac disease (gluten sensitive enteropathy), NSAID-induced gastrointestinal damage, cancer treatment induced tissue damage (e.g. chemotherapy induced enteritis), Parkinson’s disease, parenteral nutrition induced mucosal atrophy, intestinal failure in preborn infants, necrotizing enterocolitis, neonatal feeding intolerance, congenital diarrhoeal diseases, congenital or acquired digestion and absorption disorders, tissue damage induced by vascular obstruction, trauma or ischemia.

A further aspect of the invention is a method for treating the symptoms of, or treating rare congenital diarrheal diseases in a patient in need thereof, by delivering a GLP-2/GLP-1 analogue of the present invention in a therapeutically effective amount. Persistent uncontrolled diarrhoea can cause severe dehydration, electrolyte imbalance, malnutrition and failure to thrive which, if left untreated, could lead to life threatening condition including death.

In a further aspect, the present invention provides the use of a compound as outlined above for the preparation of a medicament for the treatment of Tufting enteropathy, a rare congenital diarrhoeal disease characterised by early onset severe and intractable diarrhoea that often leads to intestinal failure.

A further aspect of the invention is a method for treating metabolic diseases and syndromes in a patient in need thereof, by delivering a GLP-2/GLP-1 analogue of the present invention in a therapeutically effective amount, In one embodiment metabolic disease and syndromes include obesity, type 2 diabetes, non-alcoholic fatty liver disease (NAFLD), nonalcoholic steatohepatitis (NASH), insulin resistance, hyperglycemia, insulin resistance, glucose intolerance. It is envisaged that treatment with a GLP-2/GLP-1 analogue may restore glycemic control and insulin sensitivity. This could be beneficial for the management of hyperglycemia during enteral and parenteral nutrition therapy in patients with intestinal failure, insufficiency or malabsorption disorders.

### DETAILED DESCRIPTION OF THE INVENTION

This invention relates to novel compounds. The invention also relates to the use of novel compounds as agonists of GLP receptors. The invention further relates to the use of novel compounds in the manufacture of medicaments for use as GLP receptor agonists or for the treatment of gastrointestinal and metabolic disorders. The invention further relates to compounds, compositions and medicaments which are selective GLP-2 receptor agonists.

Accordingly, in one embodiment the invention provides a compound of the formula (1):

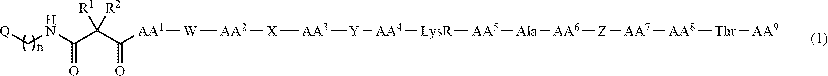

wherein;
Q is phenyl or a monocyclic heteroaryl ring each of which may be optionally substituted with one or more R^{q} groups;
R^{q} is selected from halogen, hydroxyl, amino or C₁₋₆ alkyl having an alkyl chain optionally containing one or more heteroatoms selected from O, N, or S;
n is 1 to 3;
R¹ and R² are independently selected from hydrogen or a C₁₋₆ alkyl group, or together with the carbon to which they are attached join to form a C₃₋₈ cycloalkyl or a heterocyclyl group; W is a sequence —Gly—Ser—, —Ala—Ser— or —DAla—Ser—;
X is a sequence —Ser—Asp—Glu—Nle—DPhe—Thr— or -Ser-Asp-Glu-Nle-Asn-Thr-; Y is a sequence —Leu—Asp—;
Z is a sequence -Asp-Phe-lle-Asn-Trp-Leu-lle-Gln-Thr-;
AA¹ is -NHCHR³CO-; wherein R³ is selected from -(CH₂)_{y}CONH₂, -(CH₂)_{y}COOH or -(CH₂)_{y}tetrazolyl; where y is 1 or 2;
AA² is -NHCR^{4a}R^{4b}CO-; wherein R^{4a} is hydrogen or a C₁₋₃ alkyl group; and R^{4b} is a benzyl group optionally substituted with one or more halogen groups, C₁₋₃ alkyl groups or C₁₋₃ alkoxy groups;
AA³ is -Aib- or -lle-;
AA⁴ is -NHCR^{5a}R^{5b}CO-; wherein R^{5a} is hydrogen or a C₁₋₃ alkyl group; and R^{5b} is an optionally substituted C₁₋₆ alkyl group, or -(CH₂)ₓCONH₂; where x is 1 or 2;
AA⁵ is —Ala— or -Aib-;
AA⁶ is —Lys—, -Aib- or a group —LysR—;
AA⁷ is —Lys— or —Arg—;
AA⁸ is -NHCR^{6a}R^{6b}CO-, wherein R^{6a} is hydrogen or a C₁₋₃ alkyl group; and R^{6b} is an optionally substituted C₁₋₆ alkyl group;
AA⁹ is -NHCR^{7a}R^{7b}CO-; wherein R^{7a} is hydrogen or a C₁₋₃ alkyl group; and R^{7b} is -(CH₂)_{z}COOH, or a benzyl group optionally substituted with one or more halogen groups, C₁₋₃ alkyl groups or C₁₋₃ alkoxy groups; where z is 1 or 2;
LysR is an N-substituted Lysine residue;
wherein the AA⁹ C-terminus is a carboxyl group or a carboxamide group, or is adjoined to any natural or non-natural amino acid sequence or any other moiety, functional group or groups;
or a tautomeric or stereochemically isomeric form thereof or a prodrug, salt or zwitterion thereof.

Q can be an imidazole ring. Q can be:

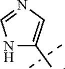

n can be 1. n can be 2. n can be 3.

R¹ and R² may be independently selected from hydrogen or a C₁₋₆ alkyl group. R¹ can be hydrogen or a C₁₋₆ alkyl group. R² can be hydrogen or a C₁₋₆ alkyl group. R¹ and R² can both be methyl. R¹ can be methyl. R² can be methyl.

W can be —Gly—Ser—. W can be —Ala—Ser—. W can be —DAla—Ser—.

X can be —Ser—Asp—Glu—Nle—DPhe—Thr—. X can be -Ser-Asp-Glu-Nle-Asn-Thr-.

AA¹ can be -NHCHR³CO-; wherein R³ is -(CH₂)_{y}tetrazolyl, where y is 1. AA¹ can be -NHCHR³CO-; wherein R³ is -(CH₂)_{y}tetrazolyl, where y is 2.

R³ can be -CH₂tetrazolyl.

AA¹ can be -NHCHR³CO-; wherein R³ is -(CH₂)_{y}COOH, where y is 1. AA¹ can be -NHCHR³CO-; wherein R³ is -(CH₂)_{y}COOH, where y is 2.

R³ can be —CH₂COOH.

AA¹ can be

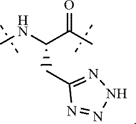

AA¹ can be —Asp—. AA¹ can be an aspartic acid residue. AA¹ can be

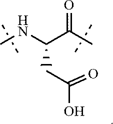

AA² can be -NHCR^{4a}R^{4b}CO-; wherein R^{4a} is hydrogen and R^{4b} is benzyl. AA² can be -NHCR^{4a}R^{4b}CO-; wherein R^{4a} is methyl and R^{4b} is benzyl. AA² can be -NHCR^{4a}R^{4b}CO-; wherein R^{4a} is methyl and R^{4b} is benzyl optionally substituted with fluorine. AA² can be -NHCR^{4a}R^{4b}CO-; wherein R^{4a} is methyl and R^{4b} is 2-fluorobenzyl.

R^{4a} can be hydrogen or methyl. R^{4a} can be hydrogen. R^{4a} can be methyl. R^{4b} can be benzyl.

R^{4b} can be benzyl optionally substituted with fluorine. R^{4b} can be 2-fluorobenzyl.

AA² can be —Phe—. AA² can be a phenylalanine residue. AA² can be

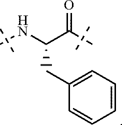

AA² can be an α-methyl phenylalanine residue. AA² can be

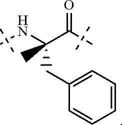

AA² can be an α-methyl 2-fluorophenylalanine residue. AA² can be

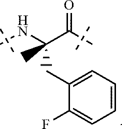

AA³ can be -Aib-. AA³ can be -lie-.

AA⁴ can be -NHCR^{5a}R^{5b}CO-; wherein R^{5a} is hydrogen and R^{5b} is isobutyl. AA⁴ can be -NHCR^{5a}R^{5b}CO-; wherein R^{5a} is methyl and R^{5b} is isobutyl. AA⁴ can be -NHCR^{5a}R^{5b}CO-; wherein R^{5a} is hydrogen and R^{5b} is -CH₂CONH₂.

R^{5a} can be hydrogen or methyl. R^{5a} can be hydrogen. R^{5a} can be methyl. R^{5b} can be isobutyl or CH₂CONH₂. R^{5b} can be isobutyl. R^{5b} can be -CH₂CONH₂.

AA⁴ can be —Leu—. AA⁴ can be a leucine residue. AA⁴ can be

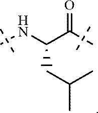

AA⁴ can be an α-methyl leucine residue. AA⁴ can be

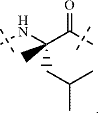

AA⁴ can be —Asn—. AA⁴ can be an asparagine residue. AA⁴ can be

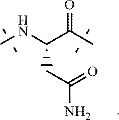

AA⁵ can be —Ala—. AA⁵ can be -Aib-.

AA⁶ can be —Lys—. AA⁶ can be -Aib-. AA⁶ can be a group —LysR—.

AA⁷ can be —Lys—. AA⁷ can be —Arg—.

AA⁸ can be -NHCR^{6a}R^{6b}CO-, wherein R^{6a} is hydrogen and R^{6b} is sec-butyl.

AA⁸ can be -NHCR^{6a}R^{6b}CO-, wherein R^{6a} is methyl and R^{6b} is isobutyl.

R^{6a} can be hydrogen or methyl. R^{6a} can be hydrogen. R^{6a} can be methyl.

R^{6b} can be isobutyl or sec-butyl. R^{6b} can be isobutyl. R^{6b} can be isobutyl.

AA⁸ can be -lle-. AA⁸ can be an isoleucine residue. AA⁸ can be

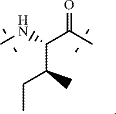

AA⁸ can be an α-methyl leucine residue. AA⁸ can be

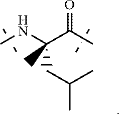

AA⁹ can be -NHCR^{7a}R^{7b}CO-; wherein R^{7a} is hydrogen and R^{7b} is -CH₂COOH. AA⁹ can be -NHCR^{7a}R^{7b}CO-; wherein R^{7a} is hydrogen and R^{7b} is benzyl. AA⁹ can be -NHCR^{7a}R^{7b}CO-; wherein R^{7a} is methyl and R^{7b} is -CH₂COOH.

R′^{a} can be hydrogen or methyl. R′^{a} can be hydrogen. R^{7a} can be methyl.

R^{7b} can be benzyl or -CH₂COOH. R^{7b} can be benzyl. R^{7b} can be -CH₂COOH.

AA⁹ can be —Asp—. AA⁹ can be an aspartic acid residue. AA⁹ can be

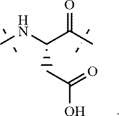

AA⁹ can be —Phe—. AA⁹ can be a phenylalanine residue. AA⁹ can be

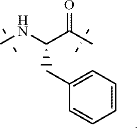

AA⁹ can be an α-methyl aspartic acid residue. AA⁹ can be

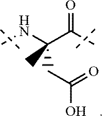

LysR can be an N-substituted Lysine residue, wherein the N-substituent is selected from: —CO(CH₂)_{q}CH₃; —CO(CH₂)_{q}CO₂H; -CO(CH₂)_{q}CHCH₂; -COO(CH₂)_{q}CH₃; -COO(CH₂)_{q}CO₂H and -COO(CH₂)_{q}CHCH₂; where q is 1 to 22.

LysR can be an N-substituted Lysine residue, wherein the N-substituent is -COO(CH₂)_{q}CHCH₂; where q is 1 to 22. LysR can be an N-substituted Lysine residue, wherein the N-substituent is -COO(CH₂)_{q}CHCH₂; where q is 1. LysR can be an N-substituted Lysine residue, wherein the N-substituent is -COOCH₂CHCH₂.

Lys R can be

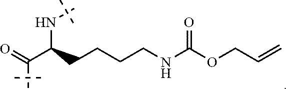

LysR can be an N-substituted Lysine residue, wherein the N-substituent is a group -L-G;
wherein L is selected from the group consisting of:

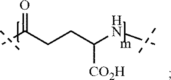

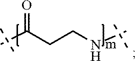

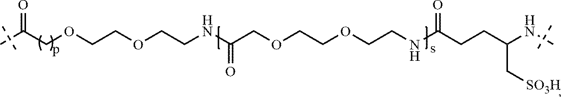

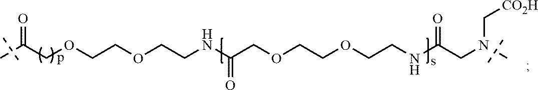

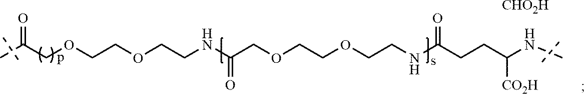

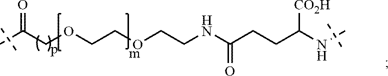

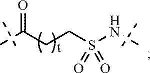

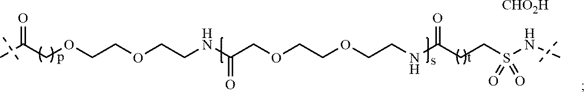

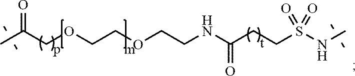

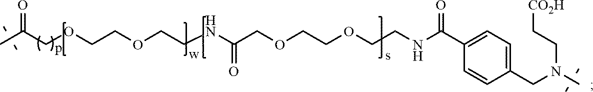

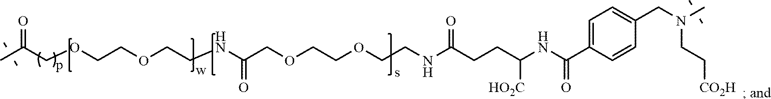

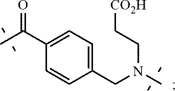

and G is selected from the group consisting of:

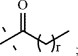

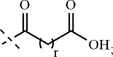

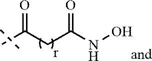

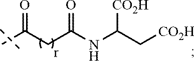

where m is 1 to 23;
p is 1 to 3;
r is 1 to 20;
s is 0 to 3;
t is 0 to 4;
and w is 0 to 4

LysR can be

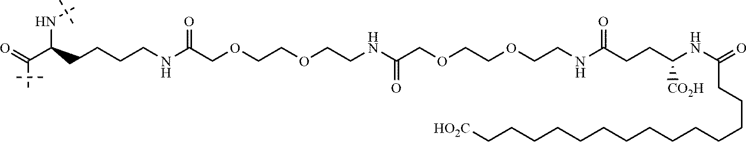

LysR can be

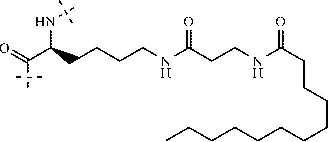

The AA⁹ C-terminus can be a carboxamide group. The AA⁹ C-terminus can be a carboxyl group. The AA⁹ C-terminus can be adjoined to any natural or non-natural amino acid sequence or any other moiety, functional group or groups.

The compound can be selected from any one of Examples 1 to 23 shown in Table 1.

Specific examples of compounds include compounds having GLP receptor agonist activity.

Specific examples of compounds include compounds having GLP-1 and/or GLP-2- receptor agonist activity.

Specific examples of compounds include compounds that have higher GLP-2 receptor agonist activity compared to GLP-1 receptor agonist activity.

The compounds of the invention may be used in a pharmaceutical composition comprising a compound of the invention and a pharmaceutically acceptable excipient.

The compounds of the invention may be used in medicine.

The present invention provides the use of a GLP-2/GLP-1 analogue compound for the preparation of a medicament for treating gastrointestinal and metabolic diseases. GLP-2/GLP-1 analogues as defined herein may be useful for promoting intestinal recovery and nutritional status of patients with malabsorption disorders, intestinal failure, intestinal insufficiency, diarrheal diseases and chronic inflammatory bowel disorders. In addition, therapeutic treatment with a GLP-2/GLP-1 analogue may improve mucosal barrier function, ameliorate gut inflammation and reduce intestinal permeability which could improve symptoms in patients with inflammatory disorders, celiac disease, congenital and acquired digestion and malabsorption syndromes, chronic diarrhoeal diseases, conditions caused by mucosal damage (e.g. cancer treatment). A GLP-2/GLP-1 analogue of the present invention is anticipated to restore glycemic control and insulin sensitivity. This could be beneficial for the management of hyperglycemia during enteral and parenteral nutrition therapy in patients with intestinal failure, insufficiency or malabsorption disorders.

In a further aspect, the present invention provides a methods of treating one of the group consisting of gastrointestinal injury, diarrheal diseases, intestinal insufficiency, intestinal failure, acid-induced intestinal injury, arginine deficiency, obesity, celiac disease, chemotherapy-induced enteritis, diabetes, obesity, fat malabsorption, steatorrhea, autoimmune diseases, food allergies, gastric ulcers, gastrointestinal barrier disorders, Parkinson’s disease, sepsis, bacterial peritonitis, inflammatory bowel disease, chemotherapy-associated tissue damage, bowel trauma, bowel ischemia, mesenteric ischemia, short bowel syndrome, malnutrition, necrotizing enterocolitis, necrotizing pancreatitis, neonatal feeding intolerance, NSAID-induced gastrointestinal damage, nutritional insufficiency, total parenteral nutrition damage to gastrointestinal tract, neonatal nutritional insufficiency, radiation-induced enteritis, radiation-induced injury to the intestines, mucositis, pouchitis, ischemia, obesity, type 2 diabetes, non-alcoholic fatty liver disease (NAFLD), nonalcoholic steatohepatitis (NASH), insulin resistance, hyperglycemia, insulin resistance, glucose intolerance.

Specifically, it is suggested that congenital diarrheal diseases which are characterised by severe diarrhoea, fluid and electrolyte loss, malabsorption and impairment of nutritional transport could be ameliorated by treatment with GLP-2 /and GLP-1 analogues of this invention. In particular, Tufting enteropathy is a condition associated with disrupted villus morphological architecture, that results in impaired nutrient absorption and enhanced intestinal permeability. Agents that can improve fluid and nutritional absorption, as well as correct the gut barrier impairment may offer value in promoting early weaning from parenteral nutrition.

Other examples of congenital diarrheal diseases that may be treated with a peptide of the invention includes brush border enzyme deficiencies (congenital lactase deficiency, congenital sucrase-isomaltase deficiency, congenital maltase-glucoamylase-deficiency), defects of membrane carriers (glucose-galactose-malabsorption, fructose malabsorption,, Acrodermatitis enteropathica, Congenital chloride I sodium diarrhoea, Primary biliary malabsorption, cystic fibrosis), lipid/lipoprotein metabolism defects (chylomicron retention disease,, abetalipoproteinemia), defects of enterocyte differentiation or cellular polarisation (Microvilious atrophy, Tufting enteropathy, Trichohepatoenteric syndrome,) and defects of enteroendocrine cells (Congenital malabsorptive diarrhoea, anendocrinosis, protein-convertase ⅓ deficiency).

The compounds of the invention may be used in the treatment of Tufting enteropathy.

### Definitions

in this application, the following definitions apply, unless indicated otherwise.

The term “alkyl”, “aryl”, “halogen”, “alkoxy”, “cycloalkyl”, “heterocyclyl” and “heteroaryl” are used in their conventional sense (e.g. as defined in the IUPAC Gold Book) unless indicated otherwise.

The term “treatment”, in relation to the uses of any of the compounds described herein, including those of the formula (1), is used to describe any form of intervention where a compound is administered to a subject suffering from, or at risk of suffering from, or potentially at risk of suffering from the disease or disorder in question. Thus, the term “treatment” covers both preventative (prophylactic) treatment and treatment where measurable or detectable symptoms of the disease or disorder are being displayed.

The term “effective therapeutic amount” as used herein (for example in relation to methods of treatment of a disorder, disease or condition) refers to an amount of the compound which is effective to produce a desired therapeutic effect. For example, if the condition is pain, then the effective therapeutic amount is an amount sufficient to provide a desired level of pain relief. The desired level of pain relief may be, for example, complete removal of the pain or a reduction in the severity of the pain.

To the extent that any of the compounds described have chiral centres, the present invention extends to all optical isomers of such compounds, whether in the form of racemates or resolved enantiomers. The invention described herein relates to all crystal forms, solvates and hydrates of any of the disclosed compounds however so prepared. To the extent that any of the compounds disclosed herein have acid or basic centres such as carboxylates or amino groups, then all salt forms of said compounds are included herein. In the case of pharmaceutical uses, the salt should be seen as being a pharmaceutically acceptable salt.

Salts or pharmaceutically acceptable salts that may be mentioned include acid addition salts and base addition salts. Such salts may be formed by conventional means, for example by reaction of a free acid or a free base form of a compound with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound in the form of a salt with another counter-ion, for example using a suitable ion exchange resin.

Examples of pharmaceutically acceptable salts include acid addition salts derived from mineral acids and organic acids, and salts derived from metals such as sodium, magnesium, potassium and calcium.

Examples of acid addition salts include acid addition salts formed with acetic, 2,2-dichloroacetic, adipic, alginic, aryl sulfonic acids (e.g. benzenesulfonic, naphthalene-2-sulfonic, naphthalene-1,5-disulfonic and p-toluenesulfonic), ascorbic (e.g. L-ascorbic), L-aspartic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, gluconic (e.g. D-gluconic), glucuronic (e.g. D-glucuronic), glutamic (e.g. L-glutamic), α-oxoglutaric, glycolic, hippuric, hydrobromic, hydrochloric, hydriodic, isethionic, lactic (e.g. (+)-L-lactic and (±)-DL-lactic), lactobionic, maleic, malic (e.g. (-)-L-malic), malonic, (i)-DL-mandetic, metaphosphoric, methanesulfonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulfuric, tannic, tartaric (e.g.(+)-L-tartaric), thiocyanic, undecylenic and valeric acids.

Also encompassed are any solvates of the compounds and their salts. Preferred solvates are solvates formed by the incorporation into the solid state structure (e.g. crystal structure) of the compounds of the invention of molecules of a non-toxic pharmaceutically acceptable solvent (referred to below as the solvating solvent). Examples of such solvents include water, alcohols (such as ethanol, isopropanol and butanol) and dimethylsulfoxide. Solvates can be prepared by recrystallising the compounds of the invention with a solvent or mixture of solvents containing the solvating solvent. Whether or not a solvate has been formed in any given instance can be determined by subjecting crystals of the compound to analysis using well known and standard techniques such as thermogravimetric analysis (TGA), differential scanning calorimetry (DSC) and X-ray crystallography.

The solvates can be stoichiometric or non-stoichiometric solvates. Particular solvates may be hydrates, and examples of hydrates include hemihydrates, monohydrates and dihydrates. For a more detailed discussion of solvates and the methods used to make and characterise them, see Bryn et al, Solid-State Chemistry of Drugs, Second Edition, published by SSCI, Inc of West Lafayette, IN, USA, 1999, ISBN 0-967-06710-3.

The term “pharmaceutical composition” in the context of this invention means a composition comprising an active agent and comprising additionally one or more pharmaceutically acceptable carriers. The composition may further contain ingredients selected from, for example, diluents, adjuvants, excipients, vehicles, preserving agents, fillers, disintegrating agents, wetting agents, emulsifying agents, suspending agents, sweetening agents, flavouring agents, perfuming agents, antibacterial agents, antifungal agents, lubricating agents and dispersing agents, depending on the nature of the mode of administration and dosage forms. The compositions may take the form, for example, of tablets, dragees, powders, elixirs, syrups, liquid preparations including suspensions, sprays, inhalants, tablets, lozenges, emulsions, solutions, cachets, granules, capsules and suppositories, as well as liquid preparations for injections, including liposome preparations.

The compounds of the invention may contain one or more isotopic substitutions, and a reference to a particular element includes within its scope all isotopes of the element. For example, a reference to hydrogen includes within its scope ¹H, ²H (D), and ³H (T). Similarly, references to carbon and oxygen include within their scope respectively ¹²C, ¹³C and ¹⁴C and ¹⁶Oand ¹⁸O. In an analogous manner, a reference to a particular functional group also includes within its scope isotopic variations, unless the context indicates otherwise. For example, a reference to an alkyl group such as an ethyl group or an alkoxy group such as a methoxy group also covers variations in which one or more of the hydrogen atoms in the group is in the form of a deuterium or tritium isotope, e.g. as in an ethyl group in which all five hydrogen atoms are in the deuterium isotopic form (a perdeuteroethyl group) or a methoxy group in which all three hydrogen atoms are in the deuterium isotopic form (a trideuteromethoxy group). The isotopes may be radioactive or non-radioactive.

Therapeutic dosages may be varied depending upon the requirements of the patient, the severity of the condition being treated, and the compound being employed. Determination of the proper dosage for a particular situation is within the skill of the art. Generally, treatment is initiated with the smaller dosages which are less than the optimum dose of the compound. Thereafter the dosage is increased by small increments until the optimum effect under the circumstances is reached. For convenience, the total daily dosage may be divided and administered in portions during the day if desired.

The magnitude of an effective dose of a compound will, of course, vary with the nature of the severity of the condition to be treated and with the particular compound and its route of administration. The selection of appropriate dosages is within the ability of one of ordinary skill in this art, without undue burden. In general, the daily dose range may be from about 10 µg to about 30 mg per kg body weight of a human and non-human animal, preferably from about 50 µg to about 30 mg per kg of body weight of a human and non-human animal, for example from about 50 µg to about 10 mg per kg of body weight of a human and non-human animal, for example from about 100 µg to about 30 mg per kg of body weight of a human and non-human animal, for example from about 100 µg to about 10 mg per kg of body weight of a human and non-human animal and most preferably from about 100 µg to about 1 mg per kg of body weight of a human and non-human animal.

### Pharmaceutical Formulations

While it is possible for the active compound to be administered alone, it is preferable to present it as a pharmaceutical composition (e.g. formulation).

Accordingly, in another embodiment of the invention, there is provided a pharmaceutical composition comprising at least one compound of the formula (1) as defined above together with at least one pharmaceutically acceptable excipient.

The composition may be a composition suitable for injection. The injection may be intravenous (IV) or subcutaneous. The composition may be supplied in a sterile buffer solution or as a solid which can be suspended or dissolved in sterile buffer for injection.

The pharmaceutically acceptable excipient(s) can be selected from, for example, carriers (e.g. a solid, liquid or semi-solid carrier), adjuvants, diluents (e.g solid diluents such as fillers or bulking agents; and liquid diluents such as solvents and co-solvents), granulating agents, binders, flow aids, coating agents, release-controlling agents (e.g. release retarding or delaying polymers or waxes), binding agents, disintegrants, buffering agents, lubricants, preservatives, anti-fungal and antibacterial agents, antioxidants, buffering agents, tonicity-adjusting agents, thickening agents, flavouring agents, sweeteners, pigments, plasticizers, taste masking agents, stabilisers or any other excipients conventionally used in pharmaceutical compositions.

The term “pharmaceutically acceptable” as used herein means compounds, materials, compositions, and/or dosage forms which are, within the scope of sound medical judgment, suitable for use in contact with the tissues of a subject (e.g. a human subject) without excessive toxicity, irritation, allergic response, or other problem or complication, commensurate with a reasonable benefit/risk ratio. Each excipient must also be “acceptable” in the sense of being compatible with the other ingredients of the formulation.

Pharmaceutical compositions containing compounds of the formula (1) can be formulated in accordance with known techniques, see for example, Remington’s Pharmaceutical Sciences, Mack Publishing Company, Easton, PA, USA.

Suitable formulations typically contain 0-20% (w/w) buffers, 0-50% (w/w) cosolvents, and/or 0-99% (wiw) Water for Injection (WFI) (depending on dose and if freeze dried). Formulations for intramuscular depots may also contain 0-99% (w/w) oils.

The compounds of the formula (1) will generally be presented in unit dosage form and, as such, will typically contain sufficient compound to provide a desired level of biological activity. For example, a formulation may contain from 1 nanogram to 2 grams of active ingredient, e.g. from 1 nanogram to 2 milligrams of active ingredient. Within these ranges, particular sub-ranges of compound are 0.1 milligrams to 2 grams of active ingredient (more usually from 10 milligrams to 1 gram, e.g. 50 milligrams to 500 milligrams), or 1 microgram to 20 milligrams (for example 1 microgram to 10 milligrams, e.g. 0.1 milligrams to 2 milligrams of active ingredient).

The active compound will be administered to a patient in need thereof (for example a human or animal patient) in an amount sufficient to achieve the desired therapeutic effect (effective amount). The precise amounts of compound administered may be determined by a supervising physician in accordance with standard procedures.

### BRIEF DESCRIPTION OF THE DRAWINGS

FIG. 1 provides a graph demonstrating the intestinal growth activity of compounds in vivo. Mice were injected subcutaneously once daily with either vehicle (0.1 % Tween80 in PBS), teduglutide or compounds (270 nmol/kg). Liraglutide was given daily as intravenous injections (200 ug/kg). Mice were sacrificed on day 3 and the wet weight of the small intestine was quantified. GLP-2 active peptides show significant enhancement of the small intestinal weight. The GLP-1 compound, liraglutide on the other hand, showed no evidence of activity in the model. Examples 1 and 3 show superior enhancements in intestinal weight compared to Teduglutide at the same doses. N=6 animals / grp. *p < 0.05 vs. vehicle

FIG. 2 provides a graph demonstrating the dose response effect of compounds on intestinal mass growth following 7 day dosing in mice. The enhancement of the small bowel wet weight (over vehicle) is plotted as a function of the peptide dose. A) Teduglutide B) Example 1 C) Example 3. Overall, Examples 1 and 3 show greater maximal enhancements in small intestinal wet weight compared to Teduglutide. N=6 animals I grp.

FIG. 3 shows the effect of Compounds or vehicle administration on the oral glucose tolerance test (OGTT). One hour before glucose challenge, Example 1 or 3 (270 nmol/kg) or vehicle (0.1% Tween80 in PBS) was administered as a single subcutaneous injection. Liraglutide was given as an iv bolus (200 ug/kg) 30 mins before oral glucose. Serial blood glucose measurements were taken at baseline and at timepoints post oral glucose challenge. (A) Time course of blood glucose levels. N=6 animals / grp. Mean +/-SEM.

### BIOLOGICAL ACTIVITY

Tables 3 and 4 provide illustration of the in vitro potency of the peptides against GLP-2R and GLP-1 R in a recombinant cell assay. The functional activity of the peptides were assessed using a HTRF cAMP assay. pEC₅₀ values are quoted. The in vitro GLP-2 assay results for compounds illustrated in Table 1 were in the range from about 0.001 nM to about 1 nM. The GLP-2 analogues of the invention demonstrate activity at both GLP-2 and GLP-1 receptors, with greater activity demonstrated at the GLP-2 receptor.

### Examples

The invention will now be illustrated, but not limited, by reference to the specific embodiments described in the following examples.

### Examples 1 to 23

The compounds of Examples 1 to 23 shown in Table 1 below have been prepared. Their LCMS properties and the methods used to prepare them are set out in Table 2. The starting materials for each of the Examples are commercial unless indicated otherwise.

TABLE 1

| | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | 25 | 26 | 27 | 28 | 29 | 30 | 31 | 32 |
|---|---|---|---|---|---|---|---|---|---|---|---|---|---|---|---|---|---|---|---|---|---|---|---|---|---|---|---|---|---|---|---|---|
| Example 1 | 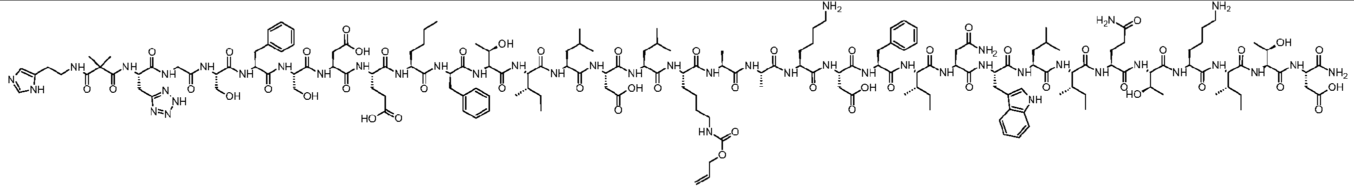 | | | | | | | | | | | | | | | | | | | | | | | | | | | | | | | |
| | CAP | TET | GLY | SER | PHE | SER | ASP | GLU | NLE | D-PHE | THR | ILE | LEU | ASP | LEU | ALloc Lys | ALA | ALA | LYS | ASP | PHE | ILE | ASN | TRP | LEU | ILE | GLN | THR | LYS | ILE | THR | ASP |
| Example 2 | 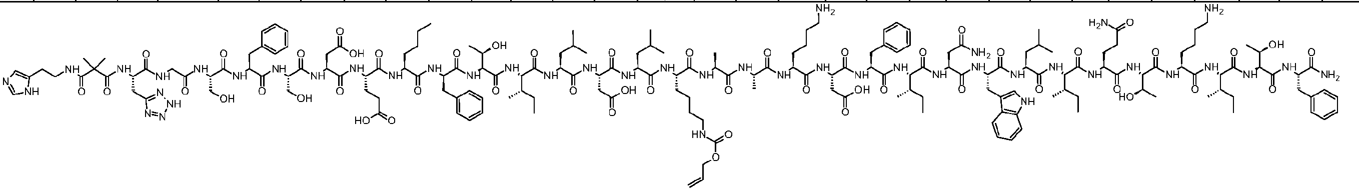 | | | | | | | | | | | | | | | | | | | | | | | | | | | | | | | |
| | CAP | TET | GLY | SER | PHE | SER | ASP | GLU | NLE | D-PHE | THR | ILE | LEU | ASP | LEU | Alloc Lys | ALA | ALA | LYS | ASP | PHE | ILE | ASN | TRP | LEU | ILE | GLN | THR | LYS | ILE | THR | PHE |
| Example 3 | 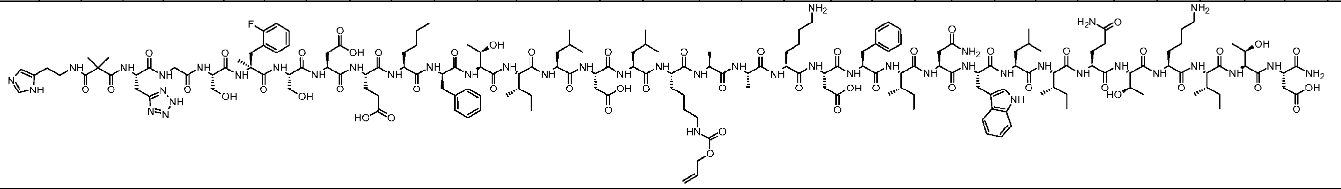 | | | | | | | | | | | | | | | | | | | | | | | | | | | | | | | |
| | CAP | TET | GLY | SER | 2–F, α-MePHE | SER | ASP | GLU | NLE | D-PHE | THR | ILE | LEU | ASP | LEU | Alloc LYS | ALA | ALA | LYS | ASP | PHE | ILE | ASN | TRP | LEU | ILE | GLN | THR | LYS | ILE | THR | ASP |
| Example 4 | 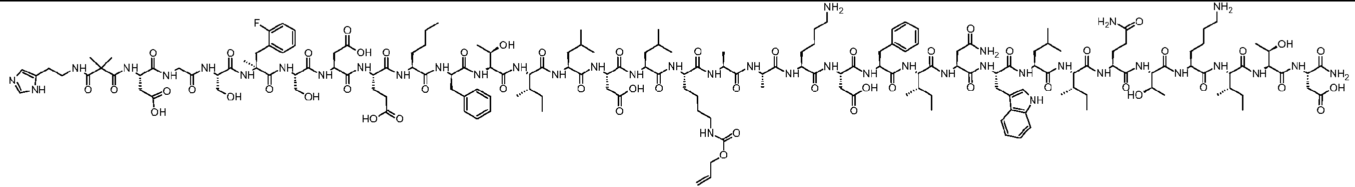 | | | | | | | | | | | | | | | | | | | | | | | | | | | | | | | |
| | CAP | ASP | GLY | SER | 2–F, α-MePHE | SER | ASP | GLU | NLE | D-PHE | THR | ILE | LEU | ASP | LEU | Alloc LYS | ALA | ALA | LYS | ASP | PHE | ILE | ASN | TRP | LEU | ILE | GLN | THR | LYS | ILE | THR | ASP |
| Example 5 | 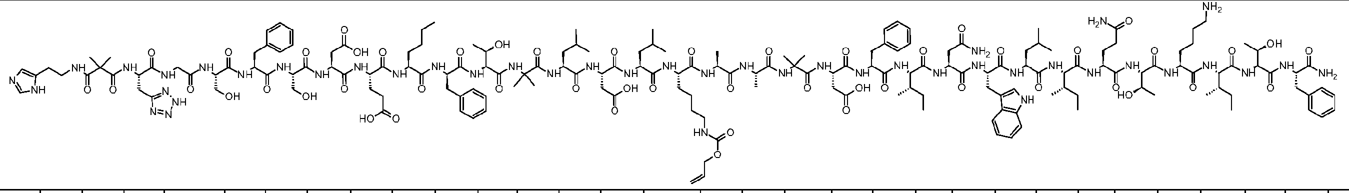 | | | | | | | | | | | | | | | | | | | | | | | | | | | | | | | |
| | CAP | TET | GLY | SER | 2–F, α-MePHE | SER | ASP | GLU | NLE | D-PHE | THR | ILE | LEU | ASP | LEU | Alloc LYS | ALA | ALA | LYS | ASP | PHE | ILE | ASN | TRP | LEU | ILE | GLN | THR | LYS | ILE | THR | PHE |
| Example 6 | 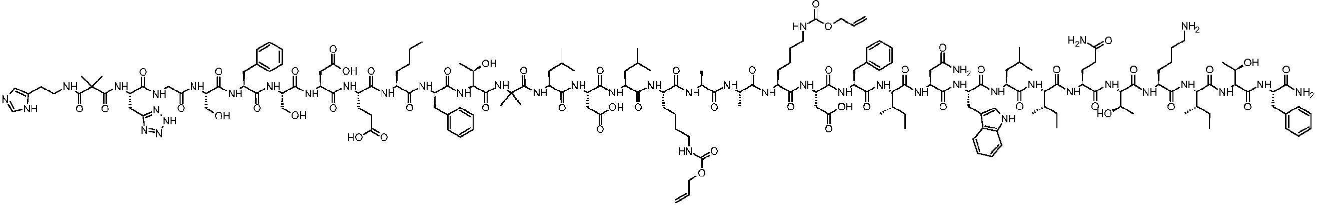 | | | | | | | | | | | | | | | | | | | | | | | | | | | | | | | |
| | CAP | TET | GLY | SER | PHE | SER | ASP | GLU | NLE | D-PHE | THR | AIB | LEU | ASP | LEU | Alloc LYS | ALA | ALA | Alloc LYS | ASP | PHE | ILE | ASN | TRP | LEU | ILE | GLN | THR | LYS | ILE | THR | PHE |
| Example 7 | 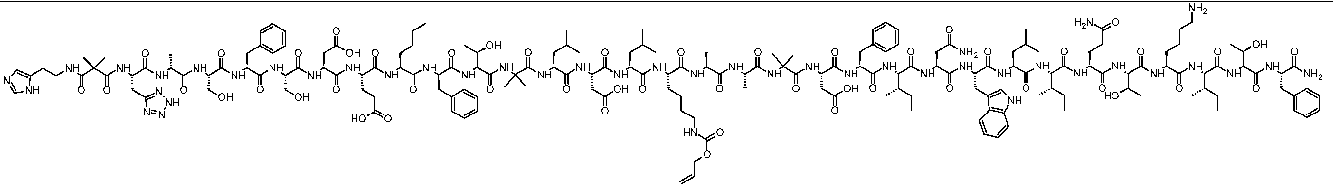 | | | | | | | | | | | | | | | | | | | | | | | | | | | | | | | |
| | CAP | TET | D-ALA | SER | PHE | SER | ASP | GLU | NLE | D-PHE | THR | AIB | LEU | ASP | LEU | Alloc LYS | ALA | ALA | AIB | ASP | PHE | ILE | ASN | TRP | LEU | ILE | GLN | THR | LYS | ILE | THR | PHE |
| Example 8 | 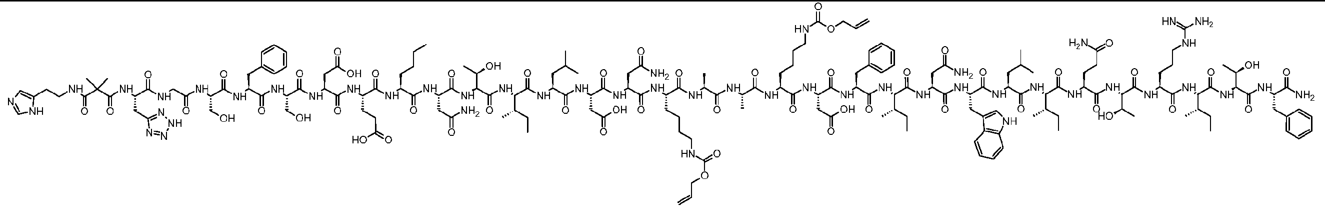 | | | | | | | | | | | | | | | | | | | | | | | | | | | | | | | |
| | CAP | TET | D-ALA | SER | PHE | SER | ASP | GLU | NLE | D-PHE | THR | AIB | LEU | ASP | LEU | Alloc LYS | ALA | ALA | Alloc LYS | ASP | PHE | ILE | ASN | TRP | LEU | ILE | GLN | THR | ARG | ILE | THR | PHE |
| Example 9 | 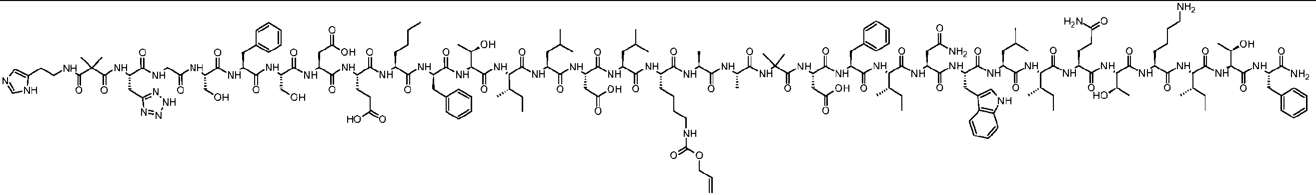 | | | | | | | | | | | | | | | | | | | | | | | | | | | | | | | |
| | CAP | TET | GLY | SER | PHE | SER | ASP | GLU | NLE | D-PHE | THR | ILE | LEU | ASP | LEU | Alloc LYS | ALA | ALA | AIB | ASP | PHE | ILE | ASN | TRP | LEU | ILE | GLN | THR | LYS | ILE | THR | PHE |
| Example 10 | 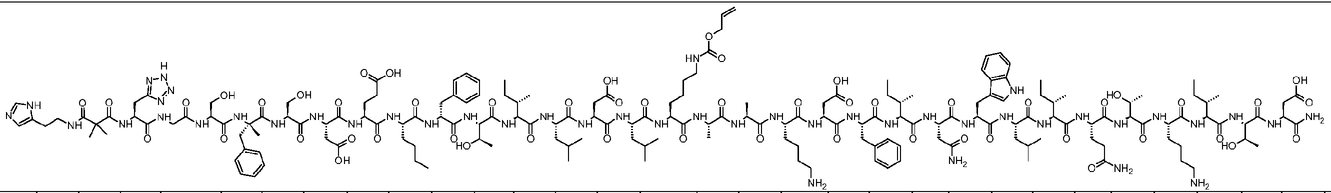 | | | | | | | | | | | | | | | | | | | | | | | | | | | | | | | |
| | CAP | TET | GLY | SER | α-MePHE | SER | ASP | GLU | NLE | D-PHE | THR | ILE | LEU | ASP | LEU | Alloc LYS | ALA | ALA | LYS | ASP | PHE | ILE | ASN | TRP | LEU | ILE | GLN | THR | LYS | ILE | THR | ASP |
| Example 11 | 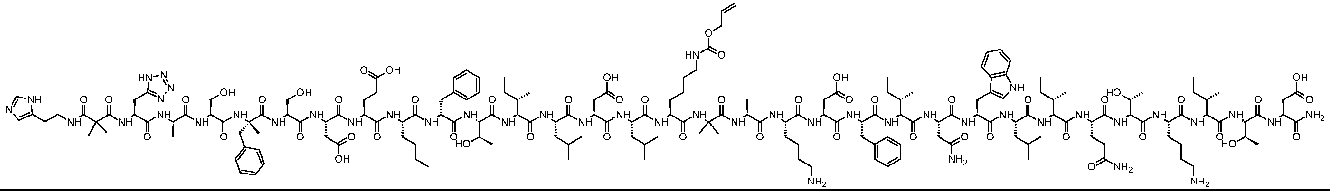 | | | | | | | | | | | | | | | | | | | | | | | | | | | | | | | |
| | CAP | TET | D-ALA | SER | α-MePHE | SER | ASP | GLU | NLE | D-PHE | THR | ILE | LEU | ASP | LEU | Alloc LYS | AIB | ALA | LYS | ASP | PHE | ILE | ASN | TRP | LEU | ILE | GLN | THR | LYS | ILE | THR | ASP |
| Example 12 | 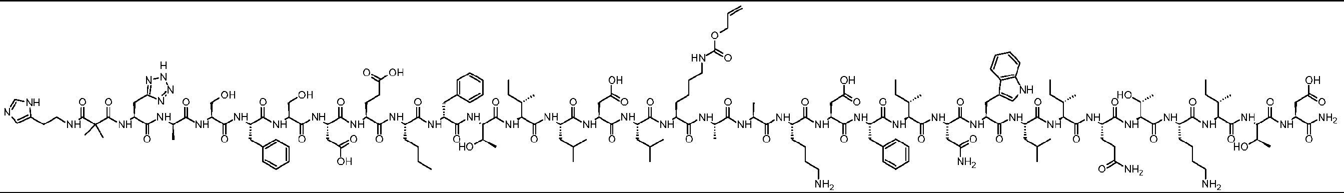 | | | | | | | | | | | | | | | | | | | | | | | | | | | | | | | |
| | CAP | TET | GLY | SER | PHE | SER | ASP | GLU | NLE | D-PHE | THR | ILE | LEU | ASP | LEU | Alloc LYS | ALA | ALA | LYS | ASP | PHE | ILE | ASN | TRP | LEU | ILE | GLN | THR | LYS | ILE | THR | ASP |
| Example 13 | 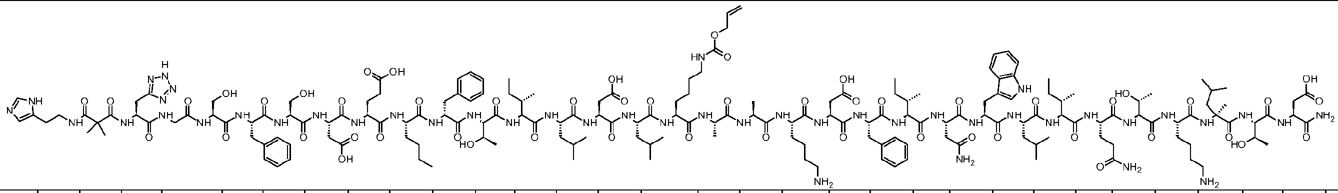 | | | | | | | | | | | | | | | | | | | | | | | | | | | | | | | |
| | CAP | TET | GLY | SER | PHE | SER | ASP | GLU | NLE | D-PHE | THR | ILE | LEU | ASP | LEU | Alloc LYS | ALA | ALA | LYS | ASP | PHE | ILE | ASN | TRP | LEU | ILE | GLN | THR | LYS | α-MeLou | THR | ASP |
| Example 14 | 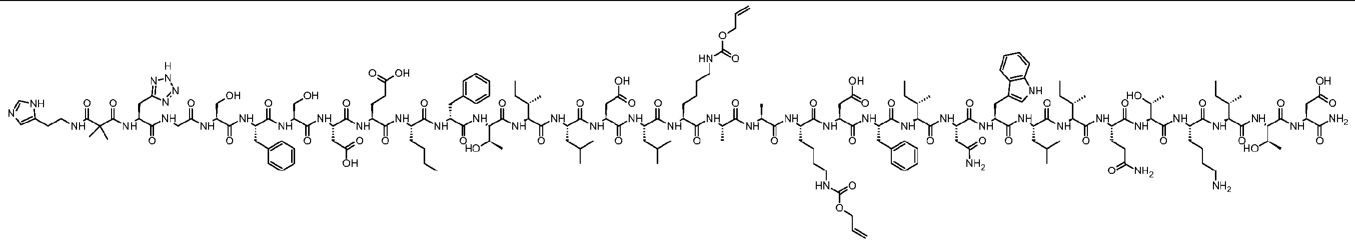 | | | | | | | | | | | | | | | | | | | | | | | | | | | | | | | |
| | CAP | TET | GLY | SER | PHE | SER | ASP | GLU | NLE | D-PHE | THR | ILE | LEU | ASP | LEU | Alloc LYS | ALA | ALA | Alloc LYS | ASP | PHE | ILE | ASN | TRP | LEU | ILE | GLN | THR | LYS | ILE | THR | ASP |
| Example 15 | 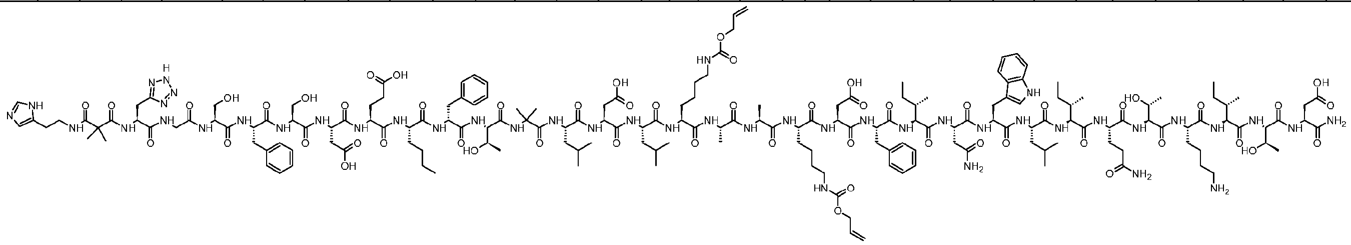 | | | | | | | | | | | | | | | | | | | | | | | | | | | | | | | |
| | CAP | TET | GLY | SER | PHE | SER | ASP | GLU | NLE | D-PHE | THR | AIB | LEU | ASP | LEU | Alloc Lys | ALA | ALA | Alloc Lys | ASP | PHE | ILE | ASN | TRP | LEU | ILE | GLN | THR | LYS | ILE | THR | ASP |
| Example 16 | 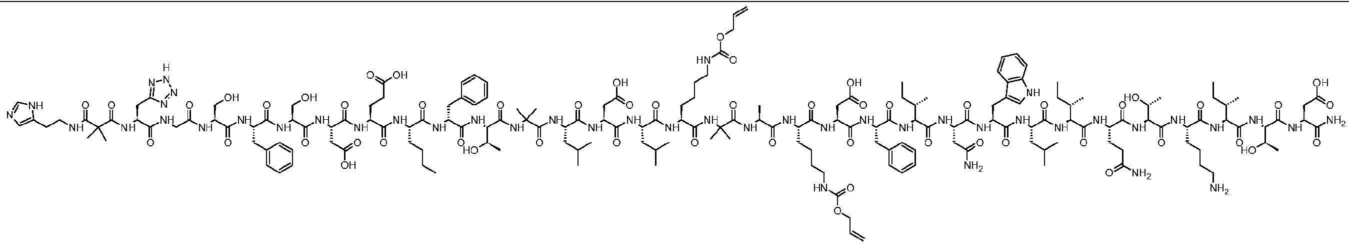 | | | | | | | | | | | | | | | | | | | | | | | | | | | | | | | |
| | CAP | TET | GLY | SER | PHE | SER | ASP | GLU | NLE | D-PHE | THR | AIB | LEU | ASP | LEU | Alloc LYS | AIB | ALA | Alloc LYS | ASP | PHE | ILE | ASN | TRP | LEU | ILE | GLN | THR | LYS | ILE | THR | ASP |
| Example 17 | 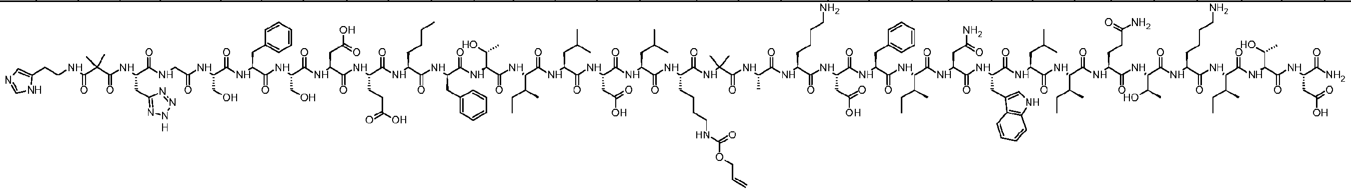 | | | | | | | | | | | | | | | | | | | | | | | | | | | | | | | |
| | CAP | TET | GLY | SER | PHE | SER | ASP | GLU | NLE | D-PHE | THR | AIB | LEU | ASP | LEU | AllocLYS | ALA | ALA | LYS | ASP | PHE | ILE | ASN | TRP | LEU | ILE | GLN | THR | LYS | ILE | THR | ASP |
| Example 18 | 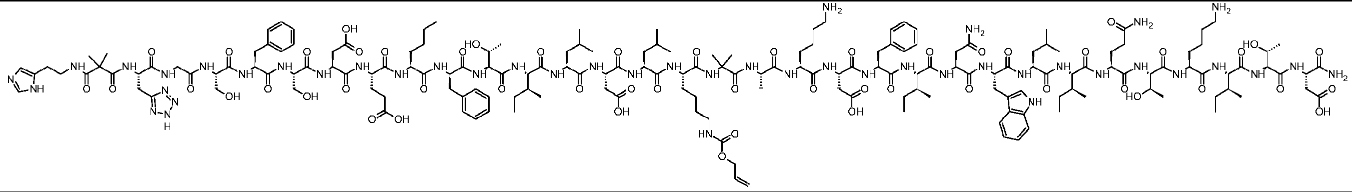 | | | | | | | | | | | | | | | | | | | | | | | | | | | | | | | |
| | CAP | TET | GLY | SER | PHE | SER | ASP | GLU | NLE | D-PHE | THR | ILE | LEU | ASP | LEU | AllocLYS | AIB | ALA | LYS | ASP | PHE | ILE | ASN | TRP | LEU | ILE | GLN | THR | LUS | ILE | THR | ASP |
| Example 19 | 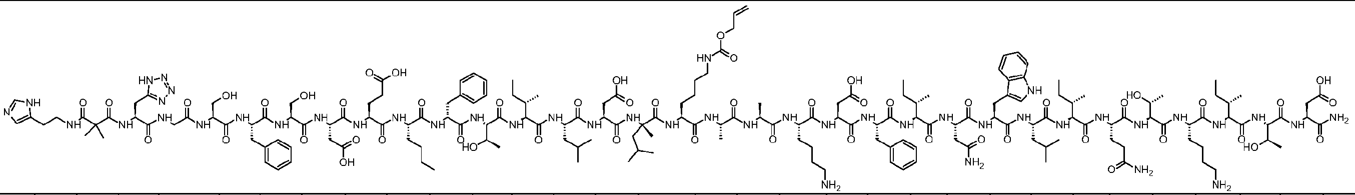 | | | | | | | | | | | | | | | | | | | | | | | | | | | | | | | |
| | CAP | TET | GLY | SER | PHE | SER | ASP | GLU | NLE | D-PHE | THR | ILE | LEU | ASP | α-MeLEU | | ALA | ALA | LYS | ASP | PHE | ILE | ASN | TRP | ALEU | ILE | GLN | THR | LYS | ILE | THR | ASP |
| Example 20 | 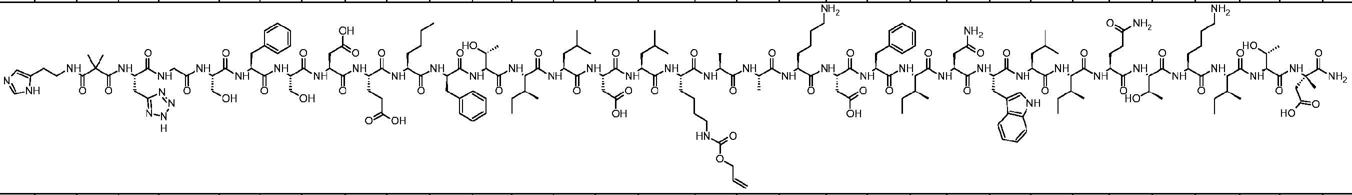 | | | | | | | | | | | | | | | | | | | | | | | | | | | | | | | |
| | CAP | TET | GLY | SER | PHE | SER | ASP | GLU | NLE | D-PHE | THR | ILE | LEU | ASP | LEU | AllocLYS | ALA | ALA | LYS | ASP | PHE | ILE | ASN | TRP | LEU | ILE | GLN | THR | LYS | ILE | THR | α-MeASP |
| Example 21 | 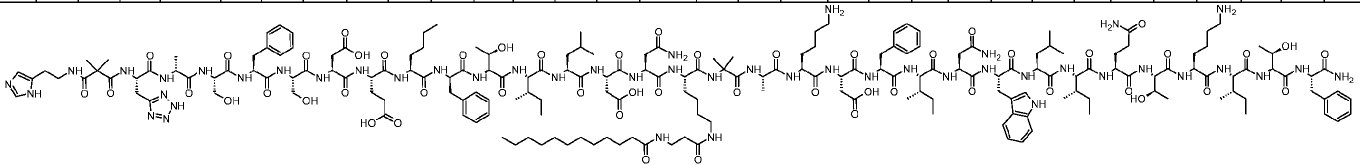 | | | | | | | | | | | | | | | | | | | | | | | | | | | | | | | |
| | CAP | TET | D-ALA | SER | PHE | SER | ASP | GLU | NLE | D-PHE | THR | ILE | LEU | ASP | ASN | LYS-β-ALA-C12 | AIB | ALA | LYS | ASP | PHE | ILE | ASN | TRP | LEU | ILE | GLN | THR | LYS | ILE | THR | PHE |
| Example 22 | 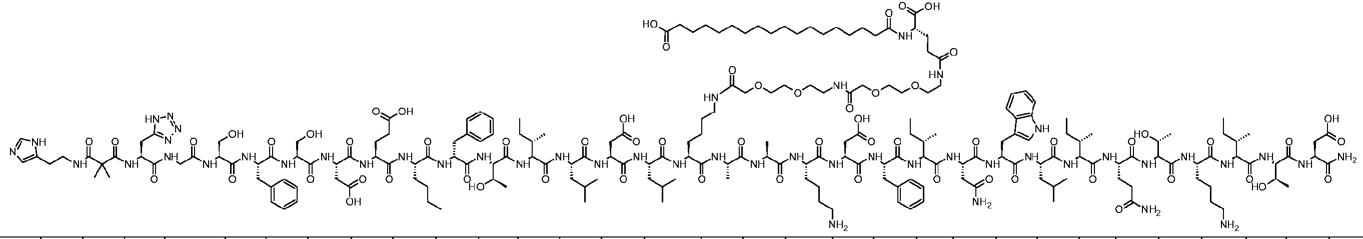 | | | | | | | | | | | | | | | | | | | | | | | | | | | | | | | |
| | CAP | TET | GLY | SER | PHE | SER | ASP | GLU | NLE | D-PHE | THR | ILE | LEU | ASP | LEU | LYS-yGlu-2xOEGC18diacid | ALA | ALA | LYS | ASP | PHE | ILE | ASN | TRP | LEU | ILE | GLN | THR | LYS | ILE | THR | ASP |
| Example 23 | 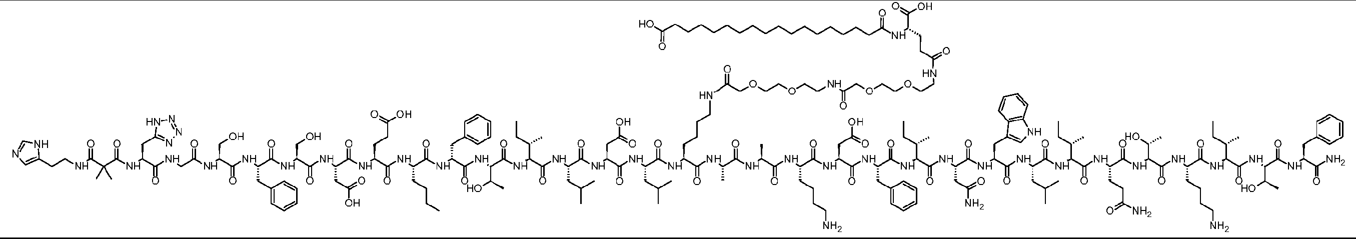 | | | | | | | | | | | | | | | | | | | | | | | | | | | | | | | |
| | CAP | TET | GLY | SER | PHE | SER | ASP | GLU | NLE | D-PHE | THR | ILE | LEU | ASP | LEU | LYS-yGlu-2xOEG C18 diacid | ALA | ALA | LYS | ASP | PHE | ILE | ASN | TRP | LEU | ILE | GLN | THR | LYS | ILE | THR | PHE |

### General Procedures

Where no preparative routes are included, the relevant intermediate is commercially available. Commercial reagents were utilized without further purification. Room temperature (rt) refers to approximately 20-27° C. ¹H NMR spectra were recorded at 400 MHz on a Bruker instrument. Chemical shift values are expressed in parts per million (ppm), i.e. (δ)-values. The following abbreviations are used for the multiplicity of the NMR signals: s=singlet, br=broad, d=doublet, t=triplet, q=quartet, quint=quintet, td=triplet of doublets, tt= triplet of triplets, qd=quartet of doublets, ddd=doublet of doublet of doublets, ddt=doublet of doublet of triplets, m=multiplet. Coupling constants are listed as J values, measured in Hz. NMR and mass spectroscopy results were corrected to account for background peaks. Chromatography refers to column chromatography performed using 60 - 120 mesh silica gel and executed under nitrogen pressure (flash chromatography) conditions.

### Analytical Methods

LCMS analysis of compounds was performed under electrospray conditions.

### LCMS Method A

Instruments: Waters Acquity UPLC, Waters 3100 PDA Detector, SQD; Column: Acquity HSS-T3, 1.8 micron, 2.1 × 100 mm; Gradient [time (min)/solvent B in A (%)]: 0.00/10, 1.00/10, 2.00/15, 4.50/55, 6.00/90, 8.00/90, 9.00/10, 10.00/10; Solvents: solvent A = 0.1% trifluoroacetic acid in water; solvent B = acetonitrile; Injection volume 1µL; Detection wavelength 214 nm; Column temperature 30° C.; Flow rate 0.3 mL per min.

### Analytical Method B

MS ion determined using LCMS method below under electrospray conditions, HPLC retention time (R_{T}) determined using HPLC method below, purity > 95% by HPLC unless indicated. LCMS: Agilent 1200 HPLC&6410B Triple Quad, Column: Xbridge C18 3.5um 2.1*30 mm. Gradient [time (min)/solvent B(%)]:0.0/10,0.9/80,1.5/90,8.5/5,1.51/10. (Solvent A=1 mL of TFA in 1000 mL Water; Solvent B=1 mL of TFA in 1000 mL of MeCN); Injection volume 5 µL (may vary); UV detection 220 nm 254 nm 210 nm; Column temperature 25° C.; 1.0 mL/min. HPLC: Agilent Technologies 1200, Column: Gemini-NX C18 5 um 110A 150*4.6 mm. Gradient [time (min)/solvent B(%)]:0.0/30,20/60,20.1/90,23/90. (Solvent A=1 mL of TFA in 1000 mL Water; Solvent B=1 mL of TFA in 1000 mL of MeCN); Injection volume 5 µL (may vary); UV detection 220 nm 254 nm; Column temperature 25° C.; 1.0 mL/min

### Analytical Method C

Instrument: Thermo Scientific Orbitrap Fusion; Column: Phenomenex Kinetex Biphenyl 100 Å, 2.6 µm, 2.1 × 50 mm; Gradient [time (min)/solvent B in A (%)]: 0.00/10, 0.30/10, 0.40/60, 1.10/90, 1.70/90, 1.75/10, 1.99/10, 2.00/10; Solvents: Solvent A = 0.1% formic acid in water; Solvent B = 0.1% formic acid in acetonitrile; Injection volume 5 µL; Column temperature 25° C.; Flow rate 0.8 mL/min.

### Synthesis of Intermediates and Compounds

The following examples are provided to illustrate preferred aspects of the invention and are not intended to limit the scope of the invention.

### Synthesis of Intermediates

All Fmoc-amino acids are commercially available except for intermediates 1 and 2

Synthesis of 2,2-dimethyl-3-oxo-3-((2-(1-trityl-1H-imidazol-4-yl)ethyl)amino)propanoic acid (Intermediate 1)

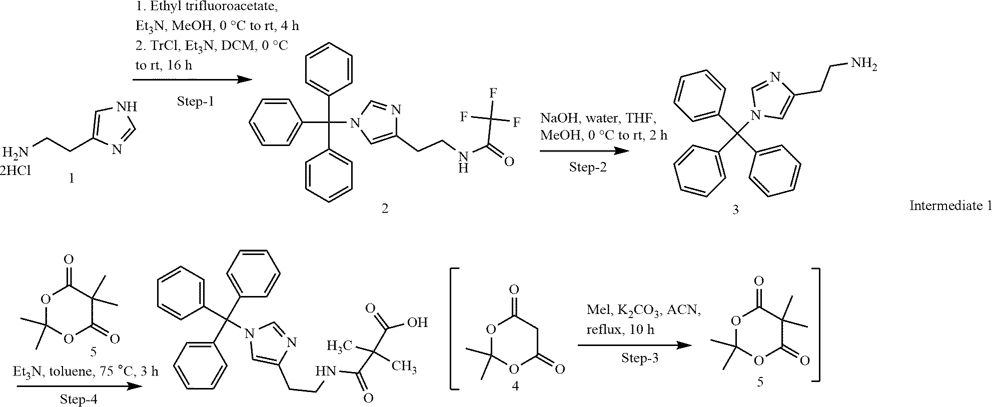

Step-1: Synthesis of 2,2,2-trifluoro-N-(2-(1-trityl-1H-imidazol-4-yl)ethyl)acetamide (2): To a solution of 2-(1H-imidazol-4-yl)ethan-1-amine dihydrochloride (1, 25.0 g, 136.6 mmol) in MeOH (100 mL), Et₃N (67 mL, 464.4 mmol) was added at rt and the reaction mixture was cooled to 0° C. A solution of ethyl trifluoroacetate (20 mL, 164.0 mmol) in MeOH (50 mL) was added to the reaction mixture over 30 min at 0° C. and the reaction mixture was stirred at rt for 4 h. This reaction mixture was diluted with dry DCM (200 mL) and Et₃N (60 mL, 409.8 mmol) and the reaction mixture was cooled to 0° C. Tr-Cl (76 g, 273.2 mmol) was added portion wise and the resulting reaction mixture was stirred at rt for 16 h. After completion, the reaction mixture was quenched with water (300 mL) and the aq layer was extracted with chloroform (3 × 150 mL). The organic layers were combined, dried (Na₂SO₄) and concentrated in vacuo. The crude residue was triturated with n-hexane to give 2,2,2-trifluoro-N-(2-(1-trityl-1H-imidazol-4-yl)ethyl)acetamide (2, 50.10 g, 81%) as a white solid.

MS (ESI +ve): 450

¹H-NMR (400 MHz; CDCl₃): δ2.75 (t, J = 5.9 Hz, 2H), 3.60 - 3.65 (m, 2H), 6.61 (s, 1H), 7.08 -7.15 (m, 6H), 7.31 - 7.38 (m, 9H), 7.40 (s, 1H), 8.41 (bs, 1H).

Step-2: Synthesis of 2-(1-trityl-1H-imidazol-4-yl)ethan-1-amine (3): To a solution of 2,2,2-trifluoro-N-(2-(1-trityl-1H-imidazol-4-yl)ethyl)acetamide (2, 50.0 g, 111.3 mmol) in THF (150 mL) and MeOH (180 mL), NaOH (22.0 g, 556.7 mmol) in water (100 mL) was slowly added at 0° C. and the reaction mixture was stirred at room temperature for 2 h. After completion, the reaction mixture was quenched with water (300 mL) and the aq layer was extracted with chloroform (3 × 150 mL). The organic layers were combined, dried (Na₂SO₄) and concentrated in vacuo to give 2-(1-trityl-1H-imidazol-4-yl)ethan-1-amine (3, 34.0 g, 86%) as a yellowish sticky solid. The crude residue was used for the next step without further purification.

MS (ESI +ve): 354

¹H-NMR (400 MHz; CDCl₃): δ 1.53 (bs, 2H), 2.65 (t, J = 6.5 Hz, 2H), 2.95 (t, J = 6.5 Hz, 2H), 6.58 (s, 1H), 7.11 - 7.16 (m, 6H), 7.28 - 7.38 (m, 10H).

Step-3: Synthesis of 2,2,5,5-tetramethyl-1,3-dioxane-4,6-dione (5): To a solution of 2,2-dimethyl-1,3-dioxane-4,6-dione (4, 20.0 g, 138.8 mmol) in ACN (200 mL), K₂CO₃ (96 g, 694.0 mmol) and Mel (26 mL, 416.6 mmol) were added at rt and reaction mixture was refluxed for 10 h. After completion, the reaction mixture was cooled to room temperature, filterd through a pad of celite, washed with EtOAc (3 × 50 mL). The organic layer was washed with 10% aq Na₂S₂O₃ (100 mL), dried, (Na₂SO₄) and concentrated in vacuo to give 2,2,5,5-tetramethyl-1,3-dioxane-4,6-dione (5, 21 g, 88%) as a yellow solid. The crude residue was used for the next step without further purification.

¹H-NMR (400 MHz; CDCl₃): δ 1.63 (s, 6H), 1.73 (s, 6H).

Step-4: Synthesis of 2,2-dimethyl-3-oxo-3-((2-(1-trityl-1H-imidazol-4-yl)ethyl)amino) propanoic acid (Intermediate 1): A solution of 2-(1-trityl-1H-imidazol-4-yl)ethan-1-amineto (3, 8.0 g, 22.6 mmol) and Et₃N (16.0 mL, 113.0 mmol) in toluene (100 mL) was added drop wise over 60 min to a solution of 2,2,5,5-tetramethyl-1,3-dioxane-4,6-dione (5, 5.8 g, 29.76 mmol) in toluene (50 mL) at 75° C. The reaction mixture was further stirred at same temperature was 3 h. After completion, the reaction mixture was concentrated in vacuo. The residue was dissolved in chloroform (100 mL) and washed with 10% aq citric acid (pH ~ 6 - 6.5). The organic layer was dried (Na₂SO₄) and concentrated in vacuo. The crude residue obtained was triturated with hot chloroform (150 mL) and n-hexane (75 mL) and the suspension was stirred at rt for 16 h. The solid was filtered, washed with chloroform : n-hexane (1:1, 2 × 50 mL) and dried in vacuo to give 2,2-dimethyl-3-oxo-3-((2-(1-trityl-1H-imidazol-4-yl)ethyl)amino)propanoic acid (Intermediate 1, 6.8 g, 64%) as a white solid.

LCMS (Method A): m/z 468 [M+H]⁺ (ES⁺), at 5.38 min, 99.31%

¹H-NMR (400 MHz; DMSO-d₆): δ 1.21 (s, 6H), 2.57 (t, J = 6.8 Hz, 2H), 3.22 - 3.27 (m, 2H), 6.66 (s, 1H), 7.06 - 7.11 (m, 6H), 7.28 (s, 1H), 7.35 - 7.42 (m, 8H), 7.64 (t, J = 5.4 Hz, 1H), 8.31 (s, 1H), 12.44 (bs, 1H).

Synthesis of (S)-2-((((9H-fluoren-9-yl)methoxy)carbonyl)amino)-3-(2-trityl-2H-tetrazol-5-yl)propanoic acid (Intermediate 2)

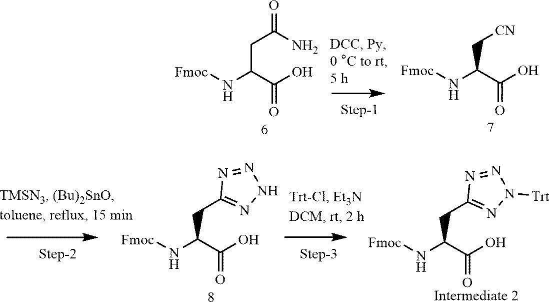

Step-1: Synthesis of (S)-2-((((9H-fluoren-9-yl)methoxy)carbonyl)amino)-3-cyanopropanoic acid (7): To a suspension of (((9H-fluoren-9-yl)methoxy)carbonyl)-L-asparagine (7, 50.0 g, 423.7 mmol) in pyridine (200 mL) was added DCC (34.0 g, 466.1 mmol) at 0° C. and the reaction mixture was stirred at room temperature for 5 h. The reaction mixture was carefully quenched with aq. 2N HCl till pH became acidic and extracted with diethyl ether (3 × 500 mL). The organic layers were combined and washed with brine, dried (Na₂SO₄) and concentrated in vacuo. The residue was triturated with pentane to give (S)-2-((((9H-fluoren-9-yl)methoxy)carbonyl)amino)-3-cyanopropanoic acid (7, 96 g, 68%) as a white solid.

MS (ESI -ve): 335.

¹H-NMR (400 MHz; DMSO-d₆): δ 2.85 - 3.05 (m, 2H), 4.22 - 4.39 (m, 4H), 7.33 (t, J = 7.6 Hz, 2H), 7.42 (t, J = 7.6 Hz, 2H), 7.72 (d, J = 7.2 Hz, 2H), 7.90 (d, J = 7.6 Hz, 2H), 8.09 (d, J = 8.4 Hz, 1H).

Step-2: Synthesis of (S)-2-((((9H-fluoren-9-yl)methoxy)carbonyl)amino)-3-(2H-tetrazol-5-yl)propanoic acid (8): To a suspension of (S)-2-((((9H-fluoren-9-yl)methoxy)carbonyl)amino)-3-cyanopropanoic acid (7, 48.0 g, 142.8 mmol) in toluene (50 mL), dibutyltin oxide (21.0 g, 85.6 mmol) was added and the reaction mixture was stirred for 15 min. To this reaction mixture trimethylsilyl azide (61 mL, 422.8 mmol) was added and reaction mixture was refluxed at 120° C. for 15 min. After cooling the reaction mixture to room temperature, the resultant solid formed was filtered and washed with diethyl ether. The solid residue was triturated with 5% MeOH/DCM (500 mL) to give (S)-2-((((9H-fluoren-9-yl)methoxy)carbonyl)amino)-3-(2H-tetrazol-5-yl)propanoic acid (8, 32.5 g, 60%) as an off white solid.

MS (ESI +ve): 380

¹H-NMR (400 MHz; DMSO-d₆): δ 3.22-3.41 (m, 2H), 4.18 - 4.28 (m, 3H), 4.41 - 4.48 (m, 1H), 7.31 (t, J = 7.2 Hz, 2H), 7.41 (t, J = 7.2 Hz, 2H), 7.65 (t, J = 7.6 Hz, 2H), 7.77 (d, J = 7.6 Hz, 1H), 7.88 (d, J = 7.6 Hz, 2H).

Step-3: Synthesis of (S)-2-((((9H-fluoren-9-yl)methoxy)carbonyl)amino)-3-(2-trityl-2H-tetrazol-5-yl)propanoic acid (Intermediate 2): To a solution of (S)-2-((((9H-fluoren-9-yl)methoxy)carbonyl)amino)-3-(2H-tetrazol-5-yl)propanoic acid (8, 12 × 5 g, 12 × 13.0 mmol) in DCM (12 × 45 mL), Et₃N (12 × 5.6 mL, 12 × 39.0 mmol) was added at 0° C. After stirring for 5 min, trityl chloride (12 × 4.0 g, 12 × 14.0 mmol) was added and the reaction mixture was stirred at the same temperature for 2 h. Reaction mixture was quenched with water (50 mL) and extracted with DCM (2 × 100 mL) (12 times). The organic layers were combined and washed with brine, dried (Na₂SO₄) and concentrated in vacuo. The residue was purified by flash column chromatography [normal phase, silica gel (100-200 mesh), gradient 1% to 5% methanol in DCM] to give (S)-2-((((9H-fluoren-9-yl)methoxy)carbonyl)amino)-3-(2-trityl-2H-tetrazol-5-yl)propanoic acid (Intermediate 2, 41 g, 41% ) as a white solid.

LCMS (Method A): m/z 620 [M-H]⁺ (ES⁻), at 5.99 min, 86.85%

¹H-NMR (400 MHz; CDCl₃): δ 3.44 - 3.62 (m, 2H), 4.12 - 4.20 (m, 1H), 4.25 - 4.32 (m, 1H), 4.36 - 4.44 (m, 1H), 4.82 - 4.88 (m, 1H), 7.02 - 7.12 (m, 6H), 7.24 - 7.32 (m, 11 H), 7.34 - 7.42 (m, 2H), 7.44 - 7.48 (m, 1H), 7.49 - 7.58 (m, 2H), 7.74 (d, J = 6.6 Hz, 2H).

Used in solid phase peptide synthesis without further purification

### Synthesis of Examples 1-23

Standard Fmoc solid phase peptide synthesis (SPPS) was used to synthesize the linear peptides which were then cleaved from the resin and purified.

### General Method for Peptide Synthesis

The peptide was synthesized using standard Fmoc chemistry.
1) Add DCM to the vessel containing Rink Amide MBHA Resin (sub: 0.35 mmol/g, 0.2 mmol, 0.57 g) and swell for 2 hours.
2) Drain and then wash with DMF (5 times, drain between each wash).
3) A solution of 20% piperidine in DMF was added agitate with N₂ bubbling for 30 min.
4) Drain and wash with DMF (5 times, drain between each wash).
5) Add Fmoc-amino acid solution (3.0 equivalents in DMF) and mix for 30 seconds, then add activation buffer (HBTU (2.85 equivalents) and DIEA (6 equivalents) in DMF), agitate with N₂ bubbling for 1 hour.
6) The coupling reaction was monitored by ninhydrin test
7) If required repeat steps 4 to 6 for same amino acid coupling if inefficient coupling occurs
8) Repeat steps 2 to 6 for next amino acid coupling.

Note: for the acids in the table below different equivalents and coupling agents were used

| Amino acid sites | Materials | Coupling reagents |
|---|---|---|
| 2 | Fmoc—Tet—OH(2.0eq) | DIC (2.0eq) and HOBt (2.0eq) |
| 1 | Cap(2.0eq) | DIC (2.0eq) and HOBt (2.0eq) |

### Peptide Cleavage and Purification

1) Add cleavage buffer (92.5%TFA/2.5%EDT/2.5%TIS/2.5%H₂O) to the flask containing the side chain protected peptide at room temperature and stir for 3 hours.

2) The peptide is precipitated with cold tert-butyl methyl ether and centrifuged (3 min at 3000 rpm).

3) Residue washed with tert-butyl methyl ether (2 times).

4) Crude peptide dried under vacuum for 2 hours.

5) The crude peptide was purified by prep-HPLC. Prep-HPLC Conditions: Instrument: Gilson 281. Solvent: A- 0.1% TFA in H2O, B- acetonitrile, Column: Luna C18 (200×25 mm; 10 µm) and Gemini C18 (150*30 mm; 5 µm) in series. Gradient [time (min)/solvent B (%)]:0.0/20, 60.0/50, 60.1/90, 70/90, 70.1/10, at 20 mL/min with UV detection (wave length = 215 nm) and then lyophilized to give Example 3 (25.8 mg, 3.1% yield).

TABLE 2

HRMS and LCMS properties of purify peptides represented by Examples 1-23

| Example | HRMS (Analytical Method C) | LCMS / HPLC (Analytical Method B) |
|---|---|---|
| 1 | HRMS (HESI/FT) m/z: [M+3H]³⁺ Calcd for C178H274N45O52 3870.9944; Found 1291.3427 | m/z 968.9 [M+4H]⁴⁺, R_{T} = 11.07 min |
| 2 | HRMS (HESI/FT) m/z: [M+3H]³⁺ Calcd for C183H278N45O50 3903.0359; Found 1302.0239 | m/z 977.2 [M+4H]⁴⁺, R_{T} = 12.99 min |
| 3 | HRMS (HESI/FT) m/z: [M+3H]³⁺ Calcd for C179H275FN45O52 3903.0007; Found 1302.0107 | m/z 976.8 [M+4H]⁴⁺, R_{T} = 12.99 min |
| 4 | HRMS (HESI/FT) m/z: [M+3H]³⁺ Calcd for C179H275FN41O54 3878.9783; Found 1294.0039 | m/z 970.8 [M+4H]⁴⁺, R_{T} = 11.16 min |
| 5 | HRMS (HESI/FT) m/z: [M+3H]³⁺ Calcd for C179H269N44O50 3831.9624; Found 1278.3325 | m/z 959.3 [M+4H]⁴⁺, R_{T} = 12.85 min |
| 6 | HRMS (HESI/FT) m/z: [M+3H]³⁺ Calcd for C185H278N45O52 3959.0259; Found 1320.6862 | m/z 991.0 [M+4H]⁴⁺, R_{T} = 12.81 min |
| 7 | HRMS (HESI/FT) m/z: [M+3H]³⁺ Calcd for C180H271 N44O50 3845.978; Found 1283.0038 | m/z 962.8 [M+4H]⁴⁺, R_{T} = 12.85 min |
| 8 | HRMS (HESI/FT) m/z: [M+3H]³⁺ Calcd for C180H274N49O54 3982.9966; Found 1328.6771 | m/z 997.0 [M+4H]⁴⁺, R_{T} = 11.80 min |
| 9 | HRMS (HESI/FT) m/z: [M+3H]³⁺ Calcd for C181H273N44O50 3859.9936; Found 1287.6761 | m/z 966.1 [M+4H]⁴⁺, R_{T} = 13.25 min |
| 10 | HRMS (HESI/FT) m/z: [M+3H]³⁺ Calcd for C179H276N45O52 3885.01; Found 1296.0151 | m/z 972.6 [M+4H]⁴⁺, R_{T} = 11.18 min |
| 11 | HRMS (HESI/FT) m/z: [M+3H]³⁺ Calcd for C181H280N45O52 3913.0415; Found 1305.3574 | m/z 979.7 [M+4H]⁴⁺, R_{T} = 12.19 min |
| 12 | HRMS (HESI/FT) m/z: [M+3H]³⁺ Calcd for C179H276N45O52 3885.01; Found 1296.0107 | m/z 972.4 [M+4H]⁴⁺, R_{T} = 10.62 min |
| 13 | HRMS (HESI/FT) m/z: [M+3H]³⁺ Calcd for C179H276N45O52 3885.01; Found 1296.0150 | m/z 1296.5 [M+3H]³⁺, R_{T} = 11.51 min |
| 14 | HRMS (HESI/FT) m/z: [M+3H]³⁺ Calcd for C182H278N45O54 3955.0156; Found 1319.3478 | m/z 1319.7 [M+3H]³⁺, R_{T} = 11.49 min |
| 15 | HRMS (HESI/FT) m/z: [M+3H]³⁺ Calcd for C180H274N45O54 3926.9844; Found 1310.0049 | m/z 1310.2 [M+3H]³⁺, R_{T} = 14.36 min |
| 16 | HRMS (HESI/FT) m/z: [M+3H]³⁺ Calcd for C181H276N45O54 3941; Found 1314.6761 | m/z 1315.3 [M+3H]³⁺, R_{T} = 14.81 min |
| 17 | HRMS (HESI/FT) m/z: [M+3H]³⁺ Calcd for C176H270N45O52 3842.9631; Found 1281.9980 | m/z 1296.5 [M+3H]³⁺, R_{T} = 14.00 min |
| 18 | HRMS (HESI/FT) m/z: [M+3H]³⁺ Calcd for C179H276N45O52 3885.01; Found 1296.0137 | m/z 1296.5 [M+3H]³⁺, R_{T} = 14.00 min |
| 19 | HRMS (HESI/FT) m/z: [M+3H]³⁺ Calcd for C179H276N45O52 3885.01; Found 1296.0170 | m/z 1296.2 [M+3H]³⁺, R_{T} = 14.16 min |
| 20 | HRMS (HESI/FT) m/z: [M+3H]³⁺ Calcd for C179H276N45O52 3885.01; Found 1296.0131 | m/z 972.4 [M+4H]⁴⁺, R_{T} = 10.79 min |
| 21 | ND | m/z 1026.7 [M+4H]⁴⁺, R_{T} = 13.48 min |
| 22 | HRMS (HESI/FT) m/z: [M+4H]⁴⁺ Calcd for C209H332N48O62 4502.3989; Found 1126.6112 | m/z 1126.8 [M+4H]⁴⁺, R_{T} = 13.39 min |
| 23 | HRMS (HESI/FT) m/z: [M+4H]⁴⁺ Calcd for C214H336N48O60 4534.4404; Found 1134.6230 | m/z 1134.8 [M+4H]⁴⁺, R_{T} = 12.13 min |

ND - Not Determined

### Biological Activity

The following examples are provided to illustrate preferred aspects of the invention and are not intended to limit the scope of the invention.

### Example A. In Vitro Pharmacological Characterization of Peptides - Functional Agonism Of Human GLP2 or GLP1 Receptors, cAMP Accumulation Assay

cAMP production upon agonist stimulation of human GLP2 or GLP1 receptor was assessed utilizing HiRange cAMP kit (Cisbio). In brief, HEK cells were infected with either human GLP2 or GLP1 receptor BacMam virus for 24 hours and frozen for later use in the assay. On the day, various concentrations of compounds were dispensed using ECHO-555 (LabCyte) to a total volume of 100 nl into a low volume 384-well Proxi plates (Perkin Elmer) followed by addition of 10µl of cell suspension delivering 800k cells per well. Cells were prepared in the assay buffer (HBSS (Lonza) supplemented with 0.5 mM IBMX (Tocris)). After 45 min incubation at 37° C., the reaction was stopped by addition of the HTRF detection reagents in the lysis buffer provided in the kit. Following 1-hour incubation at RT, plates were read on Pherastar FS (BMG Labtech, Inc.) Dotmatics Studies software was used for calculation of pEC₅₀ values by fitting data to a four parameter dose response curve.

Exendin-4 and liraglutide were used as reference compounds for GLP-1 receptor activation whilst Teduglutide and FE-203799 were used as reference compounds for GLP-2 receptor activation.

TABLE 3

| Example | Human GLP-1R agonist cAMP | | Human GLP-2R agonist cAMP | |
|---|---|---|---|---|
| | pEC₅₀ | Emax | pEC₅₀ | Emax |
| Teduglutide | 6.5 | 60.3 | 11.8 | 100.6 |
| FE-203799 | 6.8 | 61.4 | 10.8 | 100.8 |
| Exendin-4 | 12.4 | 99.7 | <8.1 | 1.6 |
| liraglutide | 11.5 | 97 | <6.1 | 1 |
| 1 | 8.5 | 94.1 | 11.0 | 102.1 |
| 2 | 8.1 | 94.7 | 10.5 | 101.0 |
| 3 | 10.1 | 99.2 | 10.8 | 102.5 |
| 4 | 9.5 | 98.1 | 10.9 | 99.3 |
| 5 | 8.1 | 93.6 | 10.9 | 103.1 |
| 6 | 8.0 | 96.4 | 10.5 | 103.0 |
| 7 | 7.4 | 62.2 | 11.0 | 102.3 |
| 8 | 7.3 | 93.3 | 10.9 | 104.1 |
| 9 | 7.7 | 94.1 | 10.9 | 102.2 |
| 10 | 10.0 | 96.4 | 11.0 | 101.0 |
| 11 | 8.8 | 95.4 | 10.9 | 101.8 |
| 12 | 7.7 | 81.1 | 11.1 | 102.2 |
| 13 | 8.6 | 93.4 | 11.1 | 100.1 |
| 14 | 7.9 | 96.1 | 11.0 | 100.8 |
| 15 | 8.3 | 95.1 | 11.1 | 101.4 |
| 16 | 7.8 | 96.0 | 11.0 | 101.5 |
| 17 | 9.1 | 93.7 | 11.3 | 100.6 |
| 18 | 8.2 | 94.0 | 11.0 | 102.4 |
| 19 | 8.6 | 92.7 | 11.0 | 102.3 |
| 20 | 8.6 | 94.0 | 11.1 | 103.2 |
| 21 | 8.1 | 91.5 | 10.4 | 99.7 |
| 22 | 8.5 | 87.6 | 10.8 | 101.0 |
| 23 | 7.9 | 89.8 | 10.5 | 102.7 |

### Example B. In Vitro Pharmacological Characterization of Peptides - Functional Agonism of Mouse GLP2 or GLP1 Receptors, cAMP Accumulation Assay

cAMP production upon agonist stimulation of mouse GLP2 or GLP1 receptors was assessed utilizing HiRange cAMP kit (Cisbio). In brief, HEK cells were transiently transfected for 24 hours with cDNA using GeneJuice Transfection reagent (EMD Millipore) and frozen at -80° C. for later use in the assay. On the day, various concentrations of compounds were dispensed using ECHO-555 (LabCyte) to a total volume of 100 nl into a low volume 384-well Proxi plate (Perkin Elmer) followed by addition of 10µl of cell suspension delivering 8000 cells per well. Cells were prepared in the assay buffer (HBSS (Lonza) supplemented with 0.5 mM IBMX (Tocris)). After 45 min incubation at 37° C., the reaction was stopped by addition of the HTRF detection reagents in the lysis buffer provided in the kit. Following 1-hour incubation at RT, plates were read on Pherastar FS (BMG Labtech, Inc.) using standard HTRF settings. Dotmatics Studies software was used for calculation of pEC50 values by fitting data to a four-parameter concentration response curve.

Liraglutide was used as reference compound for GLP-1 receptor activation whilst Teduglutide and FE-203799 were used as reference compounds for GLP-2 receptor activation.

TABLE 4

| Example | Mouse GLP-1 R agonist cAMP | | Mouse GLP-2R agonist cAMP | |
|---|---|---|---|---|
| | pEC₅₀ | Emax | pEC₅₀ | Emax |
| Teduglutide | <6.1 | 1 | 11.1 | 100.1 |
| FE-203799 | <7.3 | 53.4 | 10.5 | 98.1 |
| Iiraglutide | 11.4 | 97.9 | <6.1 | 1.6 |
| 1 | 8.5 | 95.9 | 9.7 | 102.3 |
| 2 | 7.6 | 96.1 | 8.4 | 105.5 |
| 3 | 9.4 | 99.5 | 9.3 | 103.3 |
| 12 | 7.8 | 83.3 | 9.3 | 100.2 |
| 13 | 8.5 | 92.3 | 8.4 | 101.2 |
| 15 | 8.8 | 94.4 | 8.8 | 99.9 |
| 17 | 9.2 | 94.9 | 9.1 | 100.3 |
| 20 | 8.7 | 92.2 | 9.9 | 100.4 |

### Example C: Effect on Intestinal Wet Weight in Normal Mice

C57BL/6J male mice (Charles River, Italy, ~8 weeks) are randomly allocated to the treatment group based on the baseline body weights. Animals are given free access to food and water during the whole duration of the study. Mice are dosed daily with the test compounds via subcutaneous injection. On day 4, animals are sacrificed and placed securely on a Styrofoam pad. The abdominal cavity will be opened and the intestinal tissue is excised carefully to avoid perforation. Tissue from the upper small intestine (15 cm segment from the pylorus) is collected. The bowel is cleaned by flushing through with ice cold PBS to remove any feces.

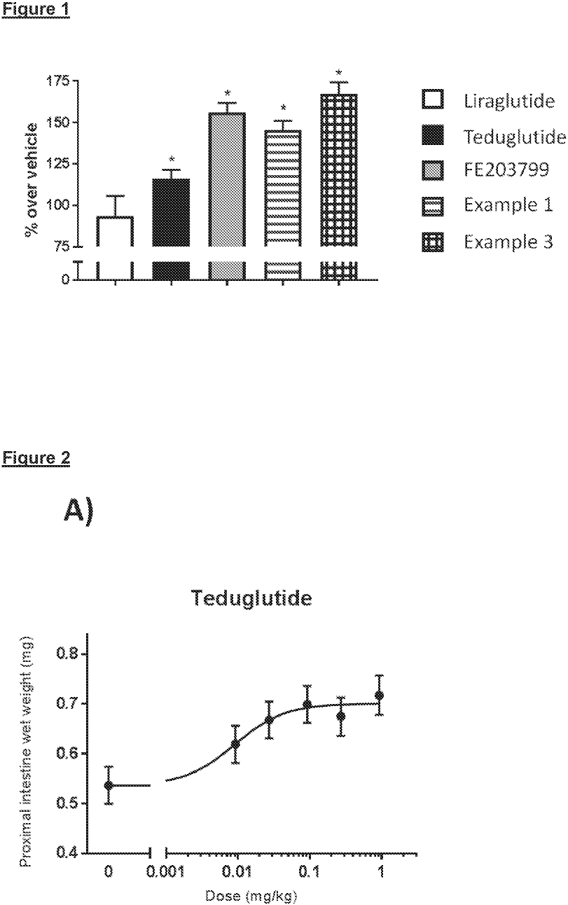

Significant enhancement of intestinal wet weight was demonstrated in animals receiving treatment with GLP-2 active peptides (teduglutide, Examples 1 and 3) while the GLP-1 peptide, liraglutide, did not enhance bowel weight (FIG. 1).

### Example D: Dose Response Effect of Compounds on Intestinal Mass in Normal Mice

C57BL/6J male mice (Charles River, Italy, ~8 weeks) are randomly allocated to the treatment group based on the baseline body weights. Animals are given free access to food and water during the whole duration of the study. Mice are dosed daily with the vehicle or test peptides via subcutaneous injection. On day 7, animals are sacrificed and segments from the upper small intestine (15 cm segment from the pylorus) are collected and weighed (see Example B).

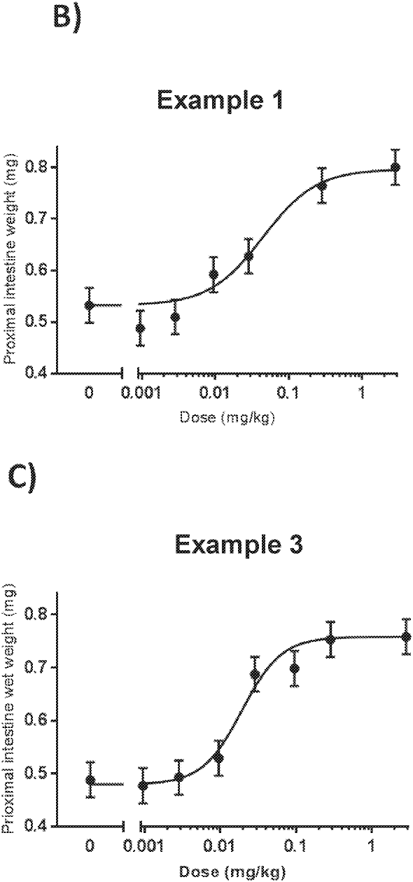

Animals receiving treatment with teduglutide or Examples 1 and 3 showed dose dependent enhancements in the wet weight of the small intestine (FIG. 2).

### Example E. Effect on Glucose Tolerance in Normal Mice

C57BL/6J male mice (Charles River, Italy, ~8w) are fasted for 6 hours on the day of testing with free access to drinking water. Prior to drug dosing, pre-dose blood glucose measurements are taken using a glucometer (ACCU-CHEK performa, Roche Diagnostic GmbH). Animals are dosed with either the vehicle (0.1% Tween80 in PBS) or compound (270 nmol/kg) via subcutaneous injection. Liraglutide (200 ug/kg) was given as an intravenous injection. 1 hour after dosing, mice are given 2 g/kg oral gavage of glucose and blood is sampled at defined time points for analysis of blood glucose levels. Timepoints of sampling: t=0 (prior to glucose dosing), 15 mins, 30 mins, 60 mins, 120 mins and 180 mins.

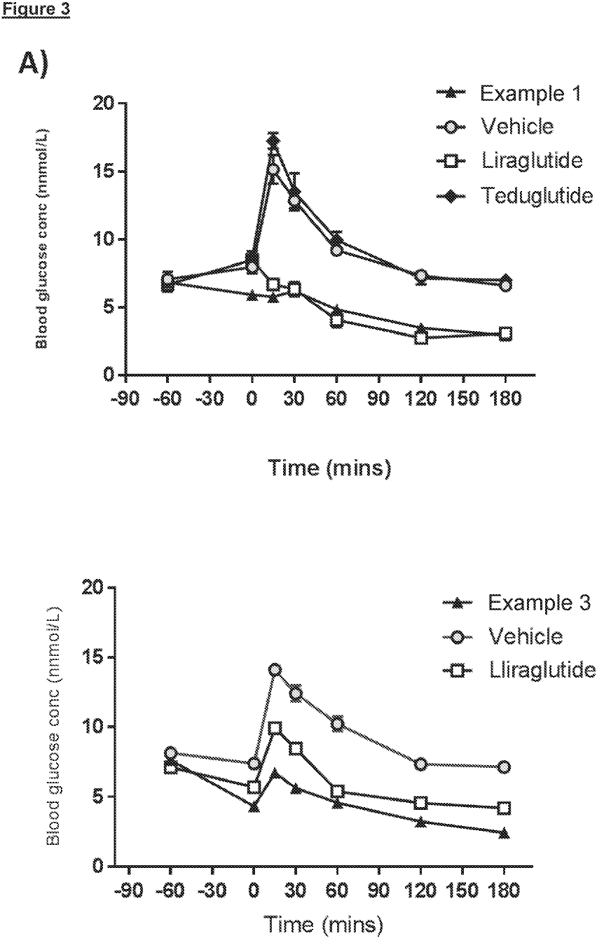

Vehicle treated mice displayed a rapid increase in blood glucose levels which reached a peak in the first 15 minutes, followed by return to baseline levels by 3 hours. In animals treated with liraglutide, Examples 1 and 3, the peak blood glucose concentration was significantly reduced (FIG. 3).

1. A compound comprising the sequence of formula (1): 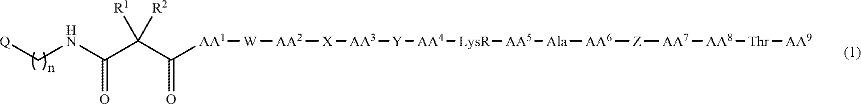
wherein;
Q is phenyl or a monocyclic heteroaryl ring each of which may be optionally substituted with one or more R^{q} groups;
R^{q} is selected from halogen, hydroxyl, amino or C₁₋₆ alkyl having an alkyl chain optionally containing one or more heteroatoms selected from O, N, or S;
n is 1 to 3;
R¹ and R² are independently selected from hydrogen or a C₁₋₆ alkyl group, or together with the carbon to which they are attached join to form a C₃₋₈ cycloalkyl or a heterocyclyl group;
W is a sequence —Gly—Ser—, —Ala—Ser— or —DAla—Ser—;
X is a sequence —Ser—Asp—Glu—Nle—DPhe—Thr— or —Ser—Asp—Glu—Nle—Asn—Thr—;
Y is a sequence —Leu—Asp—;
Z is a sequence —Asp—Phe—Ile—Asn—Trp—Leu—Ile—Gln—Thr—;
AA¹ is -NHCHR³CO-; wherein R³ is selected from -(CH₂)_{y}CONH₂, -(CH₂)_{y}COOH or -
(CH₂)_{y}tetrazolyl; where y is 1 or 2;
AA² is -NHCR^{4a}R^{4b}CO-; wherein R^{4a} is hydrogen or a C₁₋₃ alkyl group; and R^{4b} is a benzyl group optionally substituted with one or more halogen groups, C₁₋₃ alkyl groups or
C₁₋₃ alkoxy groups;
AA³ is -Aib- or —Ile—;
AA⁴ is -NHCR^{5a}R^{5b}CO-; wherein R^{5a} is hydrogen or a C₁₋₃ alkyl group; and R^{5b} is an
optionally substituted C₁₋₆ alkyl group, or -(CH₂)ₓCONH₂; where x is 1 or 2;
AA⁵ is —Ala— or -Aib-;
AA⁶ is —Lys—, -Aib- or a group —LysR—;
AA⁷ is —Lys— or —Arg—;
AA⁸ is -NHCR^{6a}R^{6b}CO-; wherein R^{6a} is hydrogen or a C₁₋₃ alkyl group; and R^{6b} is an optionally substituted C₁₋₆ alkyl group;
AA⁹ is -NHCR^{7a}R^{7b}CO-; wherein R^{7a} is hydrogen or a C₁₋₃ alkyl group; and R^{7b} is -(CH₂)_{z}COOH, or a benzyl group optionally substituted with one or more halogen groups,
C₁₋₃ alkyl groups or C₁₋₃ alkoxy groups; where z is 1 or 2;
LysR is an N-substituted Lysine residue;
wherein the AA⁹ C-terminus is a carboxyl group or a carboxamide group, or is adjoined to any natural or non-natural amino acid sequence or any other moiety, functional group or groups;
or a tautomeric or stereochemically isomeric form thereof or a prodrug, salt or zwitterion thereof.

2. The compound according to claim 1, wherein Q is: 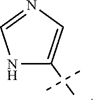
.

3. The compound according to claim 1, wherein n is 2.

4. The compound as defined in claim 1, wherein R¹ and R² are independently selected from hydrogen or a C₁₋₆ alkyl group.

5. The compound according to claim 4, wherein R¹ and R² are both methyl.

6. The compound according to claim 1, wherein R³ represents -CH₂tetrazolyl.

7. The compound according to claim 1, wherein R^{4a} is hydrogen or methyl.

8. The compound according to claim 7, wherein R^{4b} is benzyl optionally substituted with fluorine.

9. The compound according to claim 1, wherein R^{5a} is hydrogen or methyl.

10. The compound according to claim 9, wherein R^{5b} is isobutyl or -CH₂CONH₂.

11. The compound according to claim 1, wherein R^{6a} is hydrogen or methyl.

12. The compound according to claim 11, wherein R^{6b} is isobutyl or sec-butyl.

13. The compound according to claim 1, wherein R^{7a} is hydrogen or methyl.

14. The compound according to claim 13, wherein R^{7b} is benzyl or -CH₂COOH.

15. The compound according to claim 1, wherein LysR is an N-substituted Lysine residue, wherein the N-substituent is selected from: —CO(CH₂)_{q}CH₃; —CO(CH₂)_{q}CO₂H; -CO(CH₂)_{q}CHCH₂; -COO(CH₂)_{q}CH₃; -COO(CH₂)_{q}CO₂H and -COO(CH₂)_{q}CHCH₂; where q is 1 to 22.

16. The compound according to claim 1, wherein LysR is an N-substituted Lysine residue, wherein the N-substituent is a group -L-G;
wherein L is selected from the group consisting of: 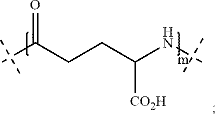 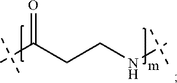 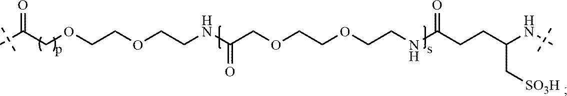 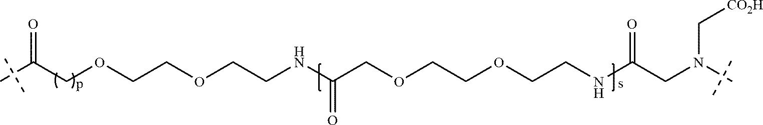 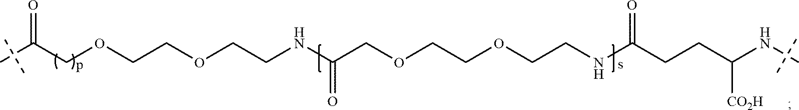 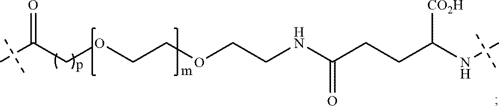 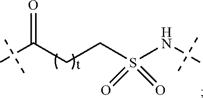 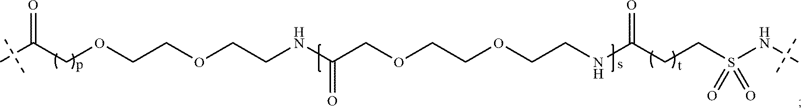 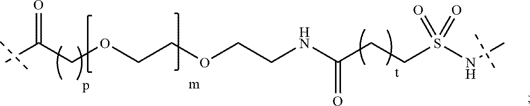 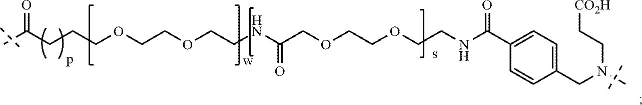 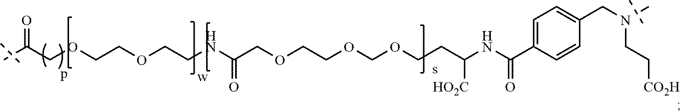
and 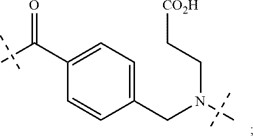
and G is selected from the group consisting of: 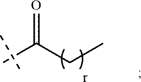 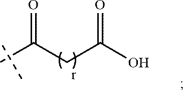 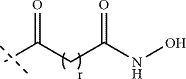
and 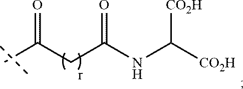
where m is 1 to 23;
p is 1 to 3;
r is 1 to 20;
s is 0 to 3;
t is 0 to 4;
and w is 0 to 4.

17. The compound according to claim 15, wherein LysR is selected from: 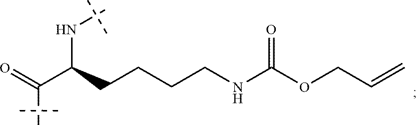 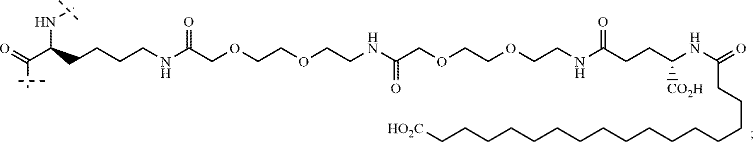
or 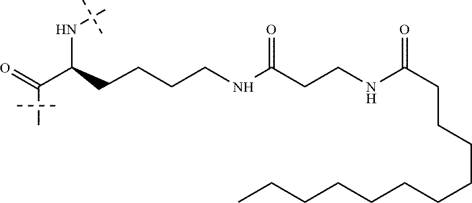
.

18. The compound according to claim 1, wherein the AA⁹ C-terminus is a carboxamide group.

19. The compound according to claim 1 which is selected from any one of Examples 1 to 23.

20. The compound according to claim 1 which is selected from: 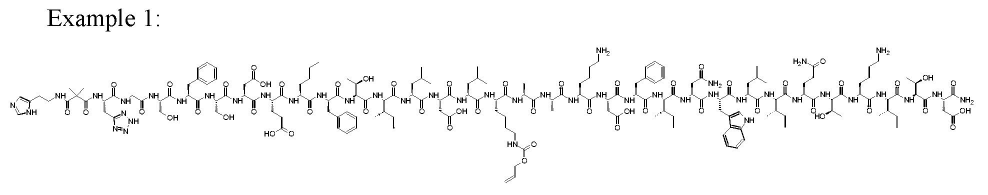 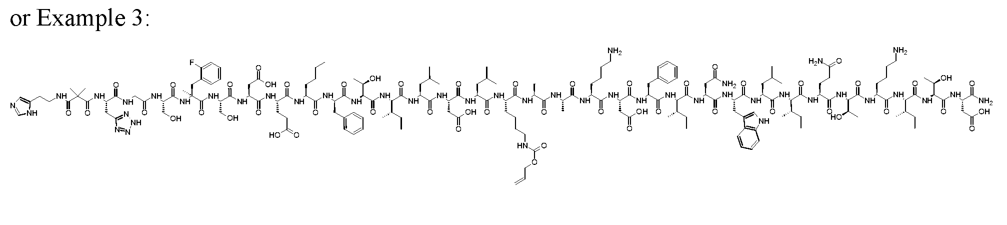 or a tautomer, salt or zwitterion thereof.

21. The compound according to claim 1 having GLP-1 and/or GLP-2 receptor agonist activity.

22. The compound according to claim 21 having higher GLP-2 receptor agonist activity compared to GLP-1 receptor agonist activity.

23. A pharmaceutical composition comprising a compound as defined in claim 1 and a pharmaceutically acceptable excipient.

24. The compound or composition according to claim 1 for use in the treatment of gastrointestinal and metabolic diseases, promoting intestinal recovery and nutritional status of patients with malabsorption disorders, intestinal failure, intestinal insufficiency, diarrheal diseases, chronic inflammatory bowel disorders, improve mucosal barrier function, ameliorate gut inflammation, inflammatory disorders, celiac disease, congenital and acquired digestion and malabsorption syndromes, chronic diarrhoeal diseases, conditions caused by mucosal damage (e.g. cancer treatment), hyperglycemia during enteral and parenteral nutrition therapy in patients with intestinal failure, insufficiency or malabsorption disorders, gastrointestinal injury, diarrheal diseases, intestinal insufficiency, intestinal failure, acid-induced intestinal injury, arginine deficiency,, obesity, celiac disease, chemotherapy-induced enteritis, diabetes, obesity, fat malabsorption, steatorrhea, autoimmune diseases, food allergies, gastric ulcers, gastrointestinal barrier disorders, Parkinson’s disease, sepsis, bacterial peritonitis, inflammatory bowel disease, chemotherapy-associated tissue damage, bowel trauma, bowel ischemia, mesenteric ischemia, short bowel syndrome, malnutrition, necrotizing enterocolitis, necrotizing pancreatitis, neonatal feeding intolerance, NSAID-induced gastrointestinal damage, nutritional insufficiency, total parenteral nutrition damage to gastrointestinal tract, neonatal nutritional insufficiency, radiation-induced enteritis, radiation-induced injury to the intestines, mucositis, pouchitis, ischemia, obesity, type 2 diabetes, non-alcoholic fatty liver disease (NAFLD), nonalcoholic steatohepatitis (NASH), insulin resistance, hyperglycemia, insulin resistance, glucose intolerance, brush border enzyme deficiencies (congenital lactase deficiency, congenital sucrase-isomaltase deficiency, congenital maltase-glucoamylase-deficiency), defects of membrane carriers (glucose-galactose-malabsorption, fructose malabsorption, Fanconi-Bickel syndrome, Acrodermatitis enteropathica, Congenital chloride / sodium diarrhoea, Lysinuric protein intolerance, Primary biliary malabsorption, cystic fibrosis), enzyme deficiencies (hereditary pancreatitis, congenital pancreas lipase deficiency), lipid/lipoprotein metabolism defects (chylomicron retention disease, hypobetalipoproteinemia, abetalipoproteinemia), defects of enterocyte differentiation or cellular polarisation (Microvillous atrophy, Tufting enteropathy, Trichohepatoenteric syndrome, Familiar haemophagocytic lymphohistiocytosis type 5), defects of enteroendocrine cells (Congenital malabsorptive diarrhoea, anendocrinosis, protein-convertase ⅓ deficiency) or congenital diarrheal diseases.

25. The use according to claim 24, wherein the disorder is Tufting enteropathy.
